SEQUENCE LISTING (28 sequences)

SEQ ID NO: 1 - protein, 24 aa, respiratory syncytial virus
  ref <301>: AUTHORS: Bastien, N Trudel, M Simard, C
  ref <302>: TITLE: Protective immune responses induced by the immunization of mice with a recombinant bacteriophage displaying an epitope of the human respiratory syncytial virus
  ref <303>: JOURNAL: Virology
  ref <304>: VOLUME: 234
  ref <306>: PAGES: 118-122
  ref <307>: DATE: 1997
MSFVPCSICSNNPTCWAICKRIPE

SEQ ID NO: 2 - DNA, 46 bp, Artificial Sequence
  Description of Artificial Sequence: Primer
gcgctcgagcatcatgtcaccctgcagcatatgcagcaacaatcca

SEQ ID NO: 3 - DNA, 41 bp, Artificial Sequence
  Description of Artificial Sequence: Primer
cgcgtcgacttgcagatagcccagcaggttggattgttgct

SEQ ID NO: 4 - DNA, 82 bp, Artificial Sequence
  Description of Artificial Sequence: Primer
gcgctcgagggtaccatgtccgccgtctacacccgaattatgatgaacggaggacgacttaagcgaccaccagaccagcttg

SEQ ID NO: 5 - DNA, 93 bp, Artificial Sequence
  Description of Artificial Sequence: Primer
cgaggtaccctcttccaccacaaggtgctcattttcgtcggatcggaagtcgtgaaggttcacaagctggtctggtggtcgcttaagtcgtcc

SEQ ID NO: 6 - protein, 40 aa, Artificial Sequence
  Description of Artificial Sequence: A chimera of an epitope G5-24 of rabies virus glycoprotein and an epitope of rabies virus nucleoprotein
MSAVYTRIMMNGGRLKRPPDQLVNLHDFRSDEIEHLVVEE

SEQ ID NO: 7 - protein, 9 aa, Artificial Sequence
  Description of Artificial Sequence: A peptide to suppress the synthesis of human growth hormone
MFCFWKTCT

SEQ ID NO: 8 - DNA, 69 bp, Artificial Sequence
  Description of Artificial Sequence: Primer
gcggaattcgtttttatttttaattttctttcaattacttccatcatgagttctttctgtttctggaaa

SEQ ID NO: 9 - DNA, 35 bp, Artificial Sequence
  Description of Artificial Sequence: Primer
gcgctcgagcgagtacacgttttccagaaacagaa

SEQ ID NO: 10 - protein, 104 aa, Artificial Sequence
  Description of Artificial Sequence: A peptide from colorectal cancer antigen GA733-2
  ref <301>: AUTHORS: Gronenborn, B Gardner, R C. Schaefer, S Shepherd, R J.
  ref <302>: TITLE: Propagation of foreign DNA in plants using cauliflower mosaic virus as vector
  ref <303>: JOURNAL: Nature
  ref <304>: VOLUME: 294
  ref <306>: PAGES: 773-776
  ref <307>: DATE: 1981
MTATFAAAQEECVCENYKLAVNCFVNNNRQCQCTSVGAQNTVCSKLAAKCLVMKAEMNGSKLGRRAKPEGALQNNDGLYDPDCDSGLFKAKQCNGTSTCWCVNT

SEQ ID NO: 11 - DNA, 38 bp, Artificial Sequence
  Description of Artificial Sequence: Primer
gcgctcgagggtaccatgcgacggcgacttttgccgca

SEQ ID NO: 12 - DNA, 34 bp, Artificial Sequence
  Description of Artificial Sequence: Primer
gtcgacctggtaccagtgttcacacaccagcacg

SEQ ID NO: 13 - DNA, 56 bp, Artificial Sequence
  Description of Artificial Sequence: Primer
gcgctcgagttcttcacaaaagaaagctggtgggaaaaggtaccgctggtaaacct

SEQ ID NO: 14 - DNA, 19 bp, Artificial Sequence
  Description of Artificial Sequence: Primer
attaaaagagctcagactc

SEQ ID NO: 15 - DNA, 54 bp, Artificial Sequence
  Description of Artificial Sequence: Primer
gcgctcgagggtaccatgtcctttgtaccctgcagcatatgcagcaacaatcca

SEQ ID NO: 16 - DNA, 54 bp, Artificial Sequence
  Description of Artificial Sequence: Primer
cgaggtaccctctggtattcttttgcagatagcccagcaggttggattgttgct

SEQ ID NO: 17 - DNA, 57 bp, Artificial Sequence
  Description of Artificial Sequence: Primer
gcgggtaccatgttctgtttctggaaaacgtgtactgctggtaaacctactaaacgt

SEQ ID NO: 18 - DNA, 32 bp, Artificial Sequence
  Description of Artificial Sequence: Primer
gcgctcgagcatcccttaggggcattcatgca

SEQ ID NO: 19 - DNA, 38 bp, Artificial Sequence; identical to SEQ ID NO: 11
  Description of Artificial Sequence: Primer

SEQ ID NO: 20 - DNA, 34 bp, Artificial Sequence; identical to SEQ ID NO: 12
  Description of Artificial Sequence: Primer

SEQ ID NO: 21 - DNA, 48 bp, Artificial Sequence
  Description of Artificial Sequence: Primer
gcactcattcaacattgctagcttatgtttttgtttacggagctcaag

SEQ ID NO: 22 - DNA, 26 bp, Artificial Sequence
  Description of Artificial Sequence: Primer
catgccattgamaggtgacacaatag

SEQ ID NO: 23 - DNA, 47 bp, Artificial Sequence
  Description of Artificial Sequence: Primer
gcgggtaccgtcgacggccagatcggccatgagtaaaggagaagaac

SEQ ID NO: 24 - DNA, 41 bp, Artificial Sequence
  Description of Artificial Sequence: Primer
gcgggcccattaatgcggccgctcatttgtatagttcatcc

SEQ ID NO: 25 - DNA, 39 bp, Artificial Sequence
  Description of Artificial Sequence: Primer
gcattaattaacatcatggtacagggcattctgtggcta

SEQ ID NO: 26 - DNA, 39 bp, Artificial Sequence
  Description of Artificial Sequence: Primer
gcactcgagttactcacttgatatgatttcatatggtct

SEQ ID NO: 27 - DNA, 45 bp, Artificial Sequence
  Description of Artificial Sequence: Primer
gcgttaattaaggccagatcggccatgggcatcaagatgggatca

SEQ ID NO: 28 - DNA, 40 bp, Artificial Sequence
  Description of Artificial Sequence: Primer
gcgttaattaagcggccgcctaacactcattcctgttgaa

(19) United States
(12) Patent Application Publication
(10) Pub. No.: US 2005/0229275 A1
(43) Pub. Date: Oct. 13, 2005

(54) Production of biomedical peptides and proteins in plants using plant virus vectors

(75) Inventors: Hilary Koprowski, Wynnewood, PA (US); Vidadi Yusibov, Havertown, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Thomas Jefferson University, Philadelphia, PA (US)

(21) Appl. No.: 11/006,071

(22) Filed: Dec. 7, 2004

Related U.S. Application Data
(63) Continuation of application No. 09/673,174, filed on Oct. 12, 2000, now abandoned.
(60) Provisional application No. 60/106,221, filed on Oct. 30, 1998.

(51) Int. Cl. A01H 1/00; C12N 15/82

(52) U.S. Cl. 800/288; 435/468

(57) ABSTRACT

The present invention discloses novel methods for producing foreign polypeptides in a host plant using recombinant viruses through functional complementation systems. The methods involve constructing suitable recombinant viral vectors that are capable of systemic infection and infecting the host plants with one or more recombinant viral vectors. The methods are also directed to infecting a host plant that is transgenic for expressing replicase genes of a virus, wherein the transgenic plant expressing a virus replicase genes complements the virus replicase function. The invention also discloses a method for producing a full-length antibody in a host plant using viral vectors.

This is a continuation of application Ser. No. 09/673,174, filed Oct. 12, 2000, which is a 371 of PCT/US99/25566, filed Oct. 29, 1999, which was published under PCT Article 21(2) in English as International Publication No. WO 00/255574, which claims the priority of U.S. Provisional Application No. 60/106,221, filed Oct. 30, 1998.

### FIELD OF THE INVENTION

The present invention is in the fields of genetic engineering and molecular farming, and especially provides methods for systemically producing foreign polypeptides including full-length antibodies in plants using recombinant viral vectors and transcomplementation systems. Also provided are recombinant full-length antibodies having higher binding affinity to the corresponding antigens compared to the parent antibodies.

### BACKGROUND OF THE INVENTION

In recent years, plants have been actively targeted for the production of medically important proteins, including vaccine antigens.

However, technical challenges remain that must be overcome before plant-based production of complex therapeutic proteins for human and animal use gains widespread acceptance in the commercial arena. Optimization of protein production levels, an important requirement to any heterologous expression system, is one of these challenges. At present, direct expression of recombinant proteins in transgenic plants does not always satisfy the requirement for high levels of protein expression. One alternative approach to expressing high levels of foreign proteins in plants is to use plant pathogens, such as plant viruses. Viral vector technology has the following advantages: (1) the flexibility to integrate and evaluate gene constructs encoding a new, improved or modified product in a few weeks time; (2) the ability to make a range of products, from peptides to complex glycosylated proteins; and (3) the demonstrated ability to achieve substantially lower cost of goods and services over alternative protein production systems.

Although plant virus-based vectors have great potential for producing foreign proteins, there is a need for improvement of several characteristics. Insertion of foreign sequences may result in failure or reduction of infectivity of virus due to interference with movement, assembly, or replication. Some of these difficulties may be circumvented by reducing the selective pressure of the host plant on virus movement and replication. The present invention accomplishes this reduction of selective pressure on the host plant by complementation of certain functions using transgenic plants and/or recombinant viruses.

### SUMMARY OF THE INVENTION

The present invention relates generally to methods and compositions for producing polypeptides in host plants using viruses. The present invention facilitates the production of desired proteins and polypeptides using transcomplementation systems involving recombinant plant viral vectors and/or transgenic plants expressing viral genes of a selected virus. Thus, the present invention is directed to a method of producing proteins and polypeptides by utilizing modified plant viruses including chimeric viruses that infect transgenic or nontrangenic plants, thereby leading to expression of the desired proteins or polypeptides throughout the plant. The terms foreign polypeptide (or protein) encoding nucleic acid sequences and heterologous nucleic acid sequences are used interchangeably herein.)

Various methods for producing foreign polypeptides in a host plant using a virus can be contemplated, as would be clear to one of ordinary skill in the art once having been apprised of the teachings.

Accordingly, the present invention, provides a method for producing a full-length antibody in a host plant using a virus. The method includes (a) constructing a first recombinant viral vector for infection which comprises a recombinant genomic component of the virus having a movement protein encoding nucleic acid sequence and a coat protein nucleic acid sequence, and a nucleic acid sequence for the heavy chain of the antibody cloned into the recombinant genomic component such that the expression of the recombinant genomic component also results in the expression of the heavy chain of the antibody;
(b) constructing a second recombinant viral vector for infection which comprises the same recombinant genomic component as in step (a) except that a nucleic acid sequence for the light chain of the antibody is cloned into the recombinant genomic component instead of the heavy chain such that the expression of the recombinant genomic component also results in the expression of the light chain of the antibody; (c) infecting the host plant at one or more locations with the first recombinant viral vector and the second recombinant viral vector such that the infection of said plant with the first and second recombinant viral vectors results in systemic infection in the host plant; (d) expressing the first and second recombinant genomic components, wherein the heavy and light chains resulting from the expression are assembled into the full-length antibody in the host plant.

The selected genomic component can be of mono-, bi-, tri-partite genomic virus. The genomic component has a movement protein encoding gene and/or a coat protein encoding gene.

At least one said foreign polypeptide-encoding nucleic acid sequence (heterologous nucleic acid sequence) which encodes a foreign polypeptide of interest is cloned into the full-length genomic component to create N-terminal fusion with the coat protein. At least one foreign polypeptide-encoding nucleic acid sequence in the recombinant viral vector is an in vitro transcription promoter sequence that is placed upstream of the remaining recombinant genomic component.

The present invention also provides method for producing foreign polypeptides in a transgenic host plant through functional transcomplementation of a virus, the method comprising: (a) constructing a recombinant viral vector for systemic infection which comprises a recombinant genomic component of the virus comprising a movement protein encoding nucleic acid sequence and a functional mutant coat protein nucleic acid sequence encoding an amino acid sequence having N-terminal deletions of up to 12 amino acids, and one or more heterologous nucleic acid sequences cloned into the recombinant genomic component wherein one of said heterologous nucleic acid sequences is fused to the N-terminus of the functional mutant coat protein nucleic acid sequence such that the expression of the recombinant genomic component also results in the expression of fused heterologous nucleic acid sequence; (b) providing said plant that is transgenic for expressing replicase genes of a virus, wherein said plant expressing said replicase genes complements the virus replicase function; (c) infecting said plant at one or more locations with the recombinant viral vector such that the infection of said plant with the recombinant viral vector at one location results in systemic infection in the host plant; and (d) producing said foreign polypeptides in said plant infected with the recombinant viral vector by growing said plant.

The amino acid sequence of the functional mutant coat protein can have 1-12 amino acids deleted from the N-terminus and the foreign polypeptide-encoding nucleic acid sequence that is fused to the N-terminus of the functional mutant coat protein. The heterologous nucleic acid sequence can encode a vaccine antigen that is selected from the group consisting of hepatitis B surface antigen, enterotoxin, rabies virus glycoprotein, rabies virus nucleoprotein, Norwalk virus capsid protein, gastrointestinal cancer antigen, G protein of Respiratory Syncytial Virus, Sandostatin or colorectal cancer antigen.

The present invention yet also provides a method for producing foreign polypeptides in a transgenic host plant through functional transcomplementation of a virus, the method comprising: (a) constructing a first recombinant viral vector for infection which comprises a recombinant genomic component of the virus comprising the native movement protein encoding nucleic acid sequence and a heterologous nucleic acid sequence in place of the native coat protein encoding nucleic acid sequence such that the expression of the recombinant genomic component also results in the expression of the heterologous nucleic acid sequence; (b) constructing a second recombinant viral vector for infection which comprises a recombinant genomic component of the virus comprising the native coat protein encoding nucleic acid sequence and a heterologous nucleic acid sequence in place of the native movement protein encoding nucleic acid sequence such that the expression of the recombinant genomic component also results in the expression of the heterologous nucleic acid sequence; (c) providing said plant that is transgenic for expressing replicase genes of a virus, wherein the transgenic plant expressing said replicase genes complements the virus replicase function; (d) infecting said plant at one or more locations with a mixture of the first recombinant viral vector and a second recombinant viral vector such that the infection of said plant with the first, second and third recombinant viral vectors at one location results in systemic infection in said plant, wherein the first recombinant viral vector expressing the native movement protein complements the cell-to-cell movement function of the virus and the second recombinant vector expressing the native coat protein complements the long distance transport function of the virus; and (e) producing said foreign polypeptides in the host plant infected with the recombinant viral vector by growing the host plant.

The present invention further provides a method for producing foreign polypeptides in a host plant through functional transcomplementation of a chimeric virus, the method comprising: (a) constructing a recombinant viral vector for systemic infection which comprises a recombinant genomic component of a first class of virus comprising a movement protein encoding nucleic acid sequence of the first class of virus, a full-length coat protein nucleic acid sequence of a second class of virus in place of the native coat protein nucleic acid sequence of the first class of virus and one or more heterologous nucleic acid sequences cloned into the recombinant genomic component such that the expression of the recombinant genomic component also results in the expression of at least one of said heterologous nucleic acid sequences; (b) infecting the host plant at one or more locations with the recombinant viral vector such that the infection of the host plant with the recombinant viral vector at one location results in systemic infection in the host plant, wherein the recombinant viral vector expressing the native movement protein of the class of virus complements the cell-to-cell movement function of the chimeric virus and that expressing the full-length coat protein nucleic acid sequence of the different class of virus complements the long distance transport function of the chimeric virus; and (c) producing said foreign polypeptides in the host plant infected with the recombinant viral vector by growing the host plant.

The present invention also provides a method for producing foreign polypeptides in a host plant through functional transcomplementation of a chimeric virus, the method comprising: (a) constructing a first recombinant viral vector for systemic infection which comprises a recombinant genomic component of a first class of virus comprising a movement protein encoding nucleic acid sequence of the first class of virus, a non-functional coat protein nucleic acid sequence of the first class of virus, a full-length coat protein nucleic acid sequence of a second class of virus inserted into the non-functional coat protein nucleic acid sequence of the first class of virus; (b) constructing a second recombinant viral vector for infection which comprises a recombinant genomic component of a class of virus comprising a movement protein encoding nucleic acid sequence of the class of virus, a non-functional coat protein nucleic acid sequence of the class of virus, and a first heterologous nucleic acid sequences cloned into the recombinant genomic component such that the expression of the recombinant genomic component also results in the expression of the first heterologous nucleic acid sequence; (c) constructing a third recombinant viral vector for systemic infection which comprises a recombinant genomic component of the first class of virus comprising a movement protein encoding nucleic acid sequence of the first class of virus, a non-functional coat protein nucleic acid sequence of the first class of virus, and a second heterologous nucleic acid sequence together with the 5′ and 3′ non-coding sequences of the full-length coat protein nucleic acid sequence of the second class of virus inserted into the non-functional coat protein nucleic acid sequence of the first class of virus such that the expression of the recombinant genomic component also results in the expression of the heterologous nucleic acid sequence; (d) infecting the host plant at one or more locations with the first, second and third recombinant viral vectors such that the infection of the host plant with said recombinant viral vectors at one location results in systemic infection in the host plant, wherein the first, second and third recombinant viral vectors expressing the native movement protein of the first class of virus complements the cell-to-cell movement function of the chimeric virus and the first recombinant viral vector expressing the full-length coat protein nucleic acid sequence of the second class of virus complements the long distance transport function of the chimeric virus; and (e) producing said foreign polypeptides in the host plant infected with the recombinant viral vector by growing the host plant.

The present invention further provides a method for eliciting an immunological response in a mammal comprising the step of: administering to the mammal an amount of a polypeptide containing plant or plant tissue thereof produced according to the methods described herein to induce an immunological response to said polypeptide in said individual.

The present invention further provides a full-length monoclonal antibody produced in a virus infected plant. The full-length monoclonal antibody has a heavy chain and a light chain, wherein the heavy chain and the light chain are assembled in planta to form the full-length monoclonal antibody, and wherein the heavy chain results from the expression of a first recombinant genomic component of the virus carrying the heavy chain gene and the light chain results from the expression of a second recombinant genomic component of the virus carrying the light chain gene in said plant.

The present invention also contemplates various compositions. Accordingly, one embodiment of the invention is a composition comprising a recombinant chimeric viral vector capable of systemic infection for producing foreign polypeptides in a host plant which comprises a recombinant genomic component of a first class of virus comprising a movement protein encoding nucleic acid sequence of the first class of virus, a coat protein nucleic acid sequence of a second class of virus in place of the native coat protein nucleic acid sequence of the first class of virus and one or more heterologous nucleic acid sequences cloned into the recombinant genomic component of the first class of virus such that the expression of the recombinant genomic component also results in the expression of at least one said heterologous nucleic acid sequence.

### BRIEF DESCRIPTION OF THE DRAWINGS

FIG. 1. Schematic representation of cloning of foreign peptides as a translational fusions of AlMV CP in full-length RNA3 of AlMV.

FIG. 2. Schematic representation of cloning of foreign peptides as a translational fusions with mutant AlMV CP (CPATG3) in full-length RNA3 of AlMV.

FIG. 3. Schematic representation showing the construction of RNA3a vectors. RNA3 is wild-type genomic RNA of AlMV.

FIG. 4. Schematic representation showing the construction of RNA3b vectors. RNA3 is wild-type genomic RNA of AlMV.

FIG. 5. Western analysis and Coomassie staining of NF1/RSV.

FIG. 6. Western analysis and Coomassie staining of NF2/RSV.

FIG. 7. Western analysis and Coomassie staining of NF2/Sand.

FIG. 8. Western analysis of NF1/TVE and NF2/TVE accumulation and assembly in infected plants.

FIG. 9. Schematic representation of the genome of Av (derivative of TMV) and construction of Av/A4 (A), Av/GFP (B), and Av/A4GFP (C): the 126 kD and 183 kD proteins are required for the TMV replication, 30 kD protein is the viral movement protein, and CP is viral coat protein.

FIG. 10. ELISA analysis of CO17-1A self-assembly in virus-infected plants.

FIG. 11. Western analysis of P3/17-1ACH expression in plants.

FIG. 12. Schematic representation of cloning (A) of genes encoding heavy chain (HC) and light chain (LC) of rAb CO17-1A and their assembly (B) in infected plant cells.

FIG. 13. ELISA analysis of CO17-1A self-assembly in virus-infected plants.

FIG. 14. Antibodies avidity measurement by competition ELISA.

FIG. 15. Effect of deglycosylation on antibody affinity measured by surface plasmon resonance.

FIG. 16. Effect of antibodies deglycosylation measured by ELISA.

### DETAILED DESCRIPTION OF THE INVENTION

The present invention is directed, among other things, to the methods for a novel means of production of recombinant foreign polypeptides and RNA sequences in plants using viruses. In particular, the embodiments of the methods disclosed herein use recombinant viral vectors which are capable of infecting a suitable host plant and systemically transcribing or expressing foreign sequences or polypeptides in the host plant.

The present invention is also directed to compositions and recombinant in vitro transcripts which are capable of systemically transcribing or expressing foreign sequences or polypeptides in a suitable host plant. Accordingly, in accordance with the subject invention, methods and compositions are provided for a novel means of production of foreign polypeptides and RNA sequences that can be easily separated from host cell components.

The practice of the present invention employs, unless otherwise indicated, conventional techniques of molecular biology, microbiology, virology, recombinant DNA technology, and immunology particularly, which are within the skill of the art. Such techniques are explained fully in the literature. All publications and references, including but not limited to patents, cited in this specification, are herein incorporated by reference in their entirety as if each individual publication or reference were specifically and individually indicated to be incorporated by reference herein as being fully set forth.

In accordance with one aspect of the subject invention, methods are provided for a novel means of production of foreign polypeptides in plants using recombinant viral vectors capable of systemically expressing foreign polypeptides upon infection. Infection as used herein is the ability of a the recombinant viral vector(s) to transfer nucleic acid to a host or introduce viral nucleic acid into a host, wherein the viral nucleic acid is replicated, both viral proteins and foreign sequences are synthesized, and new viral particles assembled having foreign sequences or proteins. The foreign polypeptide of interest in the present invention is not naturally found in the host plant.

As a general rule, the methods of the invention require constructing one or more recombinant viral vectors to carry one or more heterologous nucleic acid sequences of interest for systemic infection in the host plants. Systemic infection or the ability to spread systemically of a virus is the ability of the virus to spread from cell to cell and to replicate and express throughout the plant or in most of the cells of the plant. Thus, the ability. to introduce nucleic acid into one part of a plant for example, at one location, and have it spread to the rest of the plant would overcome the problems of growing plants from transgenic cultures. The methods of the invention can also require that after infecting a host plant with one or more recombinant viral vectors, heterologous nucleic acid sequences of interest are expressed systemically in host plants by complementation of certain functions provided by the host plants that are transgenic for certain viral genes and/or the recombinant viral vectors. The complementation functions provided by the host plants and/or the recombinant viral vectors include virus replication, assembly and movement (cell-to-cell or long distance movement). The systemic spread of foreign sequences or polypeptides through complementation functions provided by the host plants and/or the recombinant viral vectors is an essential feature of the invention. In some aspects of the invention, the recombinant viral vectors are designed such that the heterologous nucleic acid sequences are expressed systemically through transcomplementation.

Thus, the subject method includes the steps of constructing a recombinant viral vector having two or more heterologous nucleic acid sequences, infecting the host plant with the recombinant viral vector and producing foreign polypeptide of interest in the host plant by allowing the host plant to grow for some time. The process can also include isolating the desired product, if necessary. The growth of the infected host is in accordance with conventional techniques as is the isolation of the desired product. Purification of the recombinant protein, if required, is greatly simplified. The recombinant DNA or RNA encoding the polypeptide of interest can be part or all of a naturally occurring gene from any source, it may be a synthetic DNA or RNA sequence or it may be a combination of naturally occurring and synthetic sequences.

Thus, the first step in achieving any of the features of the invention is to construct a recombinant viral vector by manipulating the genomic component of a virus. Preferred virus is RNA containing plant virus. Although many plant viruses have RNA genomes, it is well known that organization of genetic information differs among groups. Thus, a virus can be a mono-, di-, tri-partite virus. “Genome” refers to the total genetic material of the virus. “RNA genome” states that as present in virions (virus particles), the genome is in RNA form.

Some of the viruses which meet this requirement, and are therefore suitable, include Alfalfa Mosaic Virus (AlMV), ilarviruses, cucumoviruses such as Cucumber Green Mottle Mosaic virus (CGMMV), closteroviruses or tobamaviruses (tobacco mosaic virus group) such as Tobacco Mosaic virus (TMV), Tobacco Etch Virus (TEV), Cowpea Mosaic virus (CMV), and viruses from the brome mosaic virus group such as Brome Mosaic virus (BMV), broad bean mottle virus and cowpea chlorotic mottle virus. Additional suitable viruses include Rice Necrosis virus (RNV), and geminiviruses such as tomato golden mosaic virus (TGMV), Cassava latent virus (CLV) and maize streak virus (MSV). Each of these groups of suitable viruses are well characterized and are well known to the skilled artisans in the field.

It should be noted that chimeric genes and vectors and recombinant plant viral nucleic acids according to this invention are constructed using techniques well known in the art. Briefly, manipulations, such as restriction, filling in overhangs to provide blunt ends, ligation of linkers, or the like, complementary ends of the fragments can be provided for joining and ligation. In carrying out the various steps, cloning is employed, so as to make the desired virus genomic component and heterologous nucleic acid combinations, to amplify the amount of DNA and to, allow for analyzing the DNA to ensure that the operations have occurred in proper manner. A wide variety of cloning vectors are available, where the cloning vector includes a replication system functional in E. coli and a marker which allows for selection of the transformed cells. Illustrative vectors include pBR332, pUC series, M13mp series, pACYC184, etc for manipulation of the primary DNA constructs. See Life Technologies Catalogue (1999); Amersham Pharmacia Biotech Catalogue (1999). Thus, the sequence may be inserted into the vector at an appropriate restriction site(s), the resulting plasmid used to transform the E. coli host, the E. coli grown in an appropriate nutrient medium and the cells harvested and lysed and the plasmid recovered. Analysis may involve sequence analysis, restriction analysis, electrophoresis, or the like. After each manipulation the DNA sequence to be used in the final construct may be restricted and joined to the next sequence, where each of the partial constructs may be cloned in the same or different plasmids. Suitable techniques have been described in standard references and well known to one skilled in the art. DNA manipulations and enzyme treatments are carried out in accordance with manufacturers' recommended procedures.

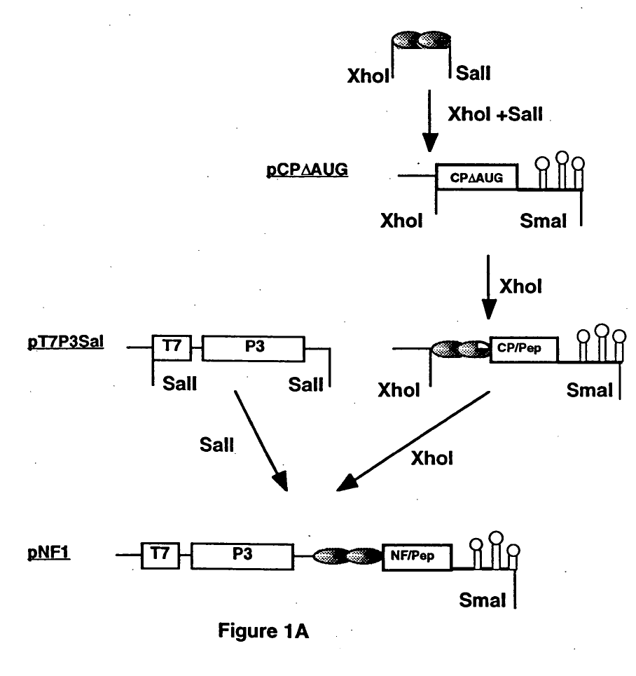

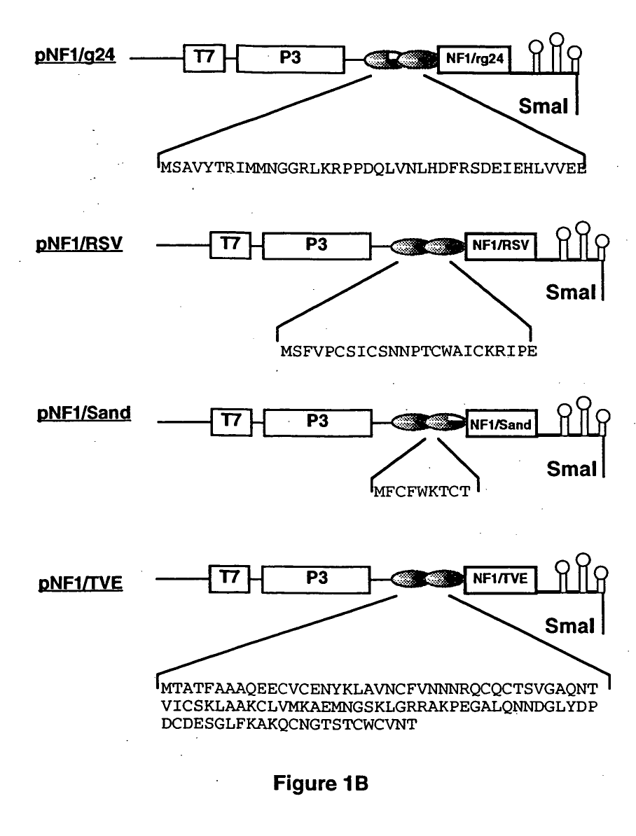

Cloning of heterologous nucleic acid sequences into the selected recombinant genomic component of the virus can take place in various ways including terminal fusions (N-terminal, C-terminal) and/or internal fusions. Construction of fusion protein requires the identification of a suitable restriction site close to the translational start codon of a gene of the viral vector, a coat protein gene for example. A suitable restriction site can be created without any alteration in coding sequence by the introduction of base changes in the start codon. As an illustration of such a modification is the AlMV coat protein shown in FIG. 1A. The AlMV coat protein here is modified in such a way that replacement of AU in AUG by TC yields an XhoI site. Alternatively, other restriction sites may be used or introduced to obtain cassette vectors that provide a convenient means to introduce heterologous nucleic and sequences encoding foreign polypeptide. The coding sequence for the foreign polypeptide can require preparation which will allow its ligation directly into the created site in the viral vector. For example, introduction of a foreign polypeptide encoding sequence into the XhoI site introduced into the AlMV coat protein described above can require the generation of compatible ends for ligation. This can typically require a single or two-base modification of site-directed mutagenesis to generate AhoI around C-terminus of the foreign peptide. The preferred method would be to use primers as linkers to produce the foreign polypeptide encoding sequence flanked by appropriate restriction sites. Orientation is checked by the use of restriction sites in the coding sequence.

The resultant construct from these N-terminal fusions would contain AlMV coat protein promoter sequence, an in-frame fusion in the first few condons of the AlMV coat protein gene of a desired foreign polypeptide-encoding sequence with its own ATG as start signal and the remainder of the AlMV protein gene sequence and terminator. Thus, protein synthesis can occur in the usual way, from the starting codon for methionine on the foreign gene to the stop codon on the viral gene (e.g., coat protein) to produce the fusion protein. In all fusions, the regulation sites on the viral genome can remain functional. Foreign polypeptide or protein-encoding nucleic acid sequence as used herein refers to the sequences that encode foreign polypeptide or protein of interest such as for example, vaccine antigen, antibodies etc.

Internal fusions involve placing of the foreign polypeptide encoding sequences or the coat protein encoding sequences of a different class of virus internally to the coding sequence of the virus, e.g., coat-protein encoding sequence. Thus, various strategies are dependent on the particular use of the nucleic acid sequence of the foreign polypeptide and would be apparent to those skilled in the art.

In some embodiments, the nucleic acid sequences encoding the foreign polypeptides or proteins (cargo peptide) are further engineered for generating recombinant polypeptides or proteins with inherent cell membrane-translocating activity in animals. Typically, in designing such a recombinant polypeptide or protein, a region of a signal peptide (used as a carrier for import into animal cells) can be placed at either the N-terminus or the C-terminus of the polypeptide of interest i.e., cargo peptide. Using this strategy it is possible to bestow membrane-translocating ability on a wide variety of proteins ranging from few amino acids to large molecular masses (over 40 kDa) to allow efficient import into living cells when administered in the animals. It is well known in the art that the hydrophobic region (h region) of a signal peptide sequence can be used as a carrier to deliver peptides (cargo) into living cells without destroying their activity. See Lin et al., 1995, J. Biol. Chem., 270: 14255-14258. Thus foreign proteins (cargo peptides), particularly vaccine antigens and antibodies can be made cell-membrane permeable simply after its attachment of fusion to a short membrane-translocating peptide sequence.

These plant produced foreign polypeptides of interest can be vaccine antigens which can be administered paventerally and/or orally. These vaccine antigens can also be engineered to fuse with proteins such as protective antigen of anthrax bacteria, heat s hock protein which can facilitate the transport of administered antigen to cell cytosol. It should be noted that the vaccine antigens can be co-expressed and co-administered together with immunoenhancers such as cytokines and hormones.

The viral coat protein gene need not encode a full-length protein; any encoded coat protein that acts as a carrier molecule of the fused protein and retains the encapsidation function is sufficient. Numerous methods are known to one skilled in the art to delete sequence from or mutate nucleic acid sequences that encode a protein and to confirm the function of the proteins encoded by these deleted or mutated sequences. Accordingly, the invention also relates to a mutated or deleted versions of a coat protein nucleic acid sequence (analogs or mutant coat proteins) of viral genomic component that encodes a protein that retains a known function. These analogs can have N-terminal, C-terminal or internal deletions, so long as function is retained. The inventors of the present invention discovered that the maximum number of amino acids that can be deleted from the N-terminus of the AlMV coat protein without altering its function is 14 amino acids. In some instances the coat protein carrying such deletions can perform significantly better than the full-length protein. Thus, in particular preferred embodiments of the invention the coat protein of a virus can have the first 10 to 12, 5 to 10, 1 to 5, 1 to 4, 3, 2 or up to 12 or up to 14 amino acid residues deleted from the N-terminus of the coat protein. In some other embodiments the first 10 to 12, 5 to 10, 1 to 5, 1 to 4, 3, 2, 1 or up to 12 or up to 14 amino acid residues of the N-terminus of the coat protein are modified or substituted in any combination. Especially preferred among these are silent substitutions and/or modifications that either enhance or do not alter the properties and activities of the resulting coat protein when present in the viral vectors used to infect plants. Deletional and modification approaches can also be applied to the other nucleic acid sequences of a virus such as the movement protein encoding sequences.

The transcription termination region which is employed will be primarily one of convenience, since in many cases termination regions appear to be relatively interchangeable. The transcription termination region is a sequence that controls formation of the 3′ end of the transcript. For example, polyadenylation sequences and self-cleaving ribozymes. The transcription termination region may be native to the transcriptional initiation region, may be native to the heterologous nucleic acid sequence encoding the polypeptide of interest, or may be derived from another source. Termination signals for expression in other organisms are well known in the literature. Sequences for accurate splicing of the transcript may also be included. Examples are introns and transposons.

Recombinant viral vectors used herein can be in vitro transcripts. After assembly of a recombinant genomic component and heterologous nucleic acid sequence(s) encoding polypeptide(s) combination, this combination can be placed behind a (downstream of) heterologous promoter (a heterologous nucleic acid sequence) that can drive in vitro transcription of the downstream sequences to produce in vitro transcripts). Examples of efficient heterologous promoters for in vitro transcription include a bacteriophage promoter such as the T7 phage promoter or SPG promoter. After such a viral vector/heterologous nucleic acid sequence(s) encoding polypeptide(s)/in vitro transcription vector combination is assembled, in vitro transcripts for infection can be produced by in vitro transcription and mixed with any other viral RNA in vitro transcripts necessary for maintenance of the viral vector in a plant cell. RNA production from the vector can be conducted, for instance, with the method described in Ausubel et al., SHORT PROTOCOLS IN MOLECULAR BIOLOGY, John Wiley & Sons, New York, 1992.

The in vitro transcripts for infection can be applied to recipient cell(s) of a plant by any of the techniques known to those skilled in the art. Suitable techniques include, but are not limited to, hand inoculations such as abrasive inoculations (leaf abrasion, abrasion in a buffer solution), mechanized spray inoculations, vacuum infiltration, particle bombardment and/or electroporation. It should be realized that the use of a mixture viral vectors can depend on the type of plant host and/or the class of virus vector used for infections. Thus in one strategy, where a AlMV virus replicase expressing transgenic plant host is used, then a mixture of recombinant viral vectors each having AlMV RNA3 or RNA4 genomic components is used. In another strategy, for example, where a non-transgenic plant host and AlMV virus as a viral vector are used, then a mixture of the recombinant viral vectors each having AlMV RNA 1, RNA2, RNA3 or RNA4 is used.

Suitable buffer solutions in which the recombinant vectors are suspended to prepare inoculum for inoculation are well known in the art. For example, leaves of plants can be inoculated with in vitro transcription products of recombinant viral vectors as described (Yusibov et. al., 1997) after adding 1 vol (v/v) of FES buffer [sodium-pyrophosphate 1% (w/v), malacoid 1% (w/v), celite 1% (w/v), glycine 0.5 M, K₂HPO₄ 0.3 M, pH 8.5, with phosphoric acid]. The mixture in vitro transcription products and FES buffer can be applied to leaves after abrading the leaf surface with carborumdum (320 grit; Fisher, Pittsburgh, Pa.). Inoculation can be affected by gentle rubbing to spread the inoculum and further abrade the leaf surface.

In preferred aspects of the invention, the initial plant inoculation can be carried out with in vitro transcripts. Once the recombinant virus particles are harvested from the host plant, these virus particles can be used as stock for further inoculations without having to use in vitro transcripts.

For embodiments of the invention wherein the foreign polypeptides are produced in plants using recombinant viral vectors, all or most of the cis-active sequences of the wild type virus which encode components necessary for production of viral particles are retained. In these embodiments heterologous nucleic acid sequences that encode foreign polypeptides are cloned into the recombinant genomic component. For example, a heterologous nucleic acid sequence that encodes a foreign polypeptide can be inserted into the recombinant genomic component of a virus having both movement protein and coat protein sequences (a situation where cis-active sequences of the wild type virus are retained).

In some instances, however, cis-active sequences which encode components necessary for production of viral particles are optionally deleted from or are rendered inactive in the recombinant viral vectors. In these instances, the missing components are supplied by complementation. For example, the missing components can be supplied by complementation in cis or in trans from a second recombinant viral vector. It is well known in the art that the term “in cis” indicates that two sequences are positioned on the same strand of RNA or DNA. The term “in trans” indicates that two sequences are positioned on different strands of RNA or DNA.

Thus, in one preferred embodiment the plant is infected with more than one recombinant viral vector (co-infection) each of which has a complementary role in the production of a viral particle. For example, the coat protein gene in a first recombinant viral vector is replaced by a heterologous nucleic acid sequence encoding a foreign polypeptide. In a second recombinant viral vector the movement protein gene is replaced by a different or same heterologous nucleic acid sequence encoding a foreign polypeptide as in the first recombinant viral vector. The first and second recombinant viral vectors can be mixed for co-infection as complementary vectors for transcomplementation. See also, the Examples. In another preferred embodiment, other functions for viral particle formation or propagation not supplied by transcomplementation described in the above embodiment are present in host plants transgenic for viral genes. For example transgenic plants expressing viral replicase genes, i.e., ReP plants, can be used to complement the virus replicase function.

An unexpected aspect of the present invention is the discovery that the coat protein gene of a first class of virus (eg., TMV) ciscomplements the long distance movement and encapsidation functions of a second class of virus (eg., AlMV) (See Example 5). Thus the complementation in the present invention can be applied rather broadly across various strains and even various genera including viruses and plants. The complementation of certain functions thus achieved has the advantage in, among other things, reducing the selective pressure by the host plant thereby facilitating the movement, assembly, or replication of the recombinant viral vectors and in extending plant host range of the recombinant viruses.

It is well known in the art the Host cells in which polypeptides including antibodies are produced have certain glycosylation capabilities. Glycosylation of antibodies (immunoglobulins) has been shown to have significant effects on their stability and affinity to binding to the corresponding antigens. By the use of the term, corresponding antigen, it is meant that the antigen that induced the formation of the antibody. Thus, an antibody is a molecule that has the particular property of combining specifically with the antigen that induced its formation.

Conventionally, antibodies are routinely produced in animals in response to antigens. In the art, antibodies are also being produced by recombinant means by using cell culture (e.g. animal cell culture). In the present invention, the recombinant production of antibodies in plants using viral vectors are particularly contemplated. The recombinant antibodies produced in plant cells can have higher binding affinity to their corresponding antigens, than the parent antibodies. By the use of the term “parent antibody”, it is meant that an antibody produced by animals in response to an antigen or an antibody that is produced recombinantly in animal cell cultures. It is particularly desired that the recombinantly produced antibody in plants has not only higher affinity to the corresponding antigen but also a stronger specific binding to the antigen. It is desired that the dissociation of the antibody produced in plants and antigen complex require higher stringency conditions than that of the parent antibody and antigen complex. Such antibodies can have a great clinical significance. Thus, plant produced antibodies with higher affinity and lower dissociation constants as compared to the parent antibodies is desired because of several advantages that can be readily recognized by those skilled in the art (e.g., it reduces the amount of antibody required to be administered to a patient and hence at a lower cost, and risk of adverse effects.

A variety of techniques are available for the genetic transformation of plants and plant tissues (i.e., the stable integration of foreign DNA into plants) and are well-known to those skilled in the art. These include transformation by Agrobacterium species and transformation by direct gene transfer. For example, the chimeric DNA constructs may be introduced into host cells obtained from dicotyledonous plants, such as tobacco and brassicas using standard Agrobacterium vectors by a transformation protocol such as that described by Moloney et al., 1989, Plant Cell Rep., 8:238-242 of Hinchee et al., 1988, Bio/Technol., 6:915-922; or other techniques known to those skilled in the art. For example, the use of T-DNA for transformation of plant cells has received extensive study and is amply described in Knauf, et al., (1983), Genetic Analysis of Host Range Expression by Agrobacterium, p. 245, In: Molecular Genetics of the Bacteria-Plant Interaction, Puhler, A. ed., Springer-Verlag, NY; Hoekema et al., (1985), Chapter V, In: The Binary Plant Vector System Offset-drukkerij Kanters B. V., Alblasserdam; and An et al., (1985), EMBO J., 4:277-284. Briefly, explants can be co-cultivated with A. tumefaciens or A. rhizogenes to allow for transfer of the transcription construct to the plant cells. Following transformation using Agrobacterium, the plant cells are dispersed in an appropriate medium for selection, subsequently callus, shoots and eventually plantlets are recovered. The Agrobacterium host will harbour a plasmid comprising the vir genes necessary for transfer of the T-DNA to the plant cells. See also, Dodds, J. ed., Plant Genetic Engineering, Cambridge University Press, Cambridge (1985).

The use of non-Agrobacterium techniques permits the use of the constructs described herein to obtain transformation and expression in a wide variety of monocotyledonous and dicotyledonous plants and other organisms. These techniques are especially useful for species that are intractable in an Agrobacterium transformation system. Other techniques for gene transfer include biolistics (Sanford, 1988, Trends in Biotech., 6:299-302), electroporation (Fromm et al., 1985, Proc. Natl. Acad. Sci. U.S.A., 82:5824-5828; Riggs and Bates, 1986, Proc. Natl. Acad. Sci. U.S.A. 82:5602-5606 or PEG-mediated DNA uptake (Potrykus et al., 1985, Mol. Gen. Genet., 199:169-177).

The foreign polypeptides of interest to be produced using viruses by any of the specific methods described herein, can be any peptide or protein. The heterologous nucleic acid sequence encoding the polypeptide of interest can be naturally derived, synthetic, or a combination thereof.

The invention is not limited by the source or the use of the recombinant polypeptide. Of particular interest are those proteins or peptides that have a biomedical, therapeutic and/or diagnostic value. These proteins or polypeptides include vaccine antigens, such as viral coat proteins or G proteins or microbial cell wall or toxin proteins, cancer antigens or various other antigenic peptides, antibodies, specifically a single-chain antibody having a translational fusion of the VH or VL chains of an immunoglobulin, peptides of direct therapeutic value such as interleukin-1, the anticoagulant hirudin and blood clotting factors. Vaccine antigens derived from pathogenic parasites such as Entamoeba and the like can also be used. Other biomedical agents such as human growth hormone or bovine somatotropin can also be produced.

In particular, the vaccine agents from the following pathogens can be particularly mentioned; S. typhi (the cause of human typhoid), S typhimurium (the case of salmonellosis), S. enteritis (a cause of food poisoning in humans), S. cholerae (the cause of salmonellosis in animals), Bordetella pertussis (the case of whooping cough), Haemophilus influenzae (a cause of meningitis), Neisseria gonorrohoeae (the cause of gonorrohoea) and Haemophilus. The vaccine agents from pathogenic parasites such as Entameoba are also included.

In accordance with the present invention, the host plants included within the scope of the present invention are all species of higher and lower plants of the Plant Kingdom. Mature plants, seedlings, and seeds are included in the scope of the invention. A mature plant includes a plant at any stage in development beyond the seedling. A seedling is a very young, immature plant in the early stages of development. Specifically, plants that can be used as hosts to produce foreign sequences and polypeptides include and are not limited to Angiosperms, Bryophytes such as Hepaticae (liverworts) and Musci (mosses); Pteridophytes such as ferns, horsetails, and lycopods; Gymnosperms such as conifers, cycads, Ginkgo, and Gnetales; and Algae including Chlorophyceae, Phaeophpyceae, Rhodophyceae, Myxophyceae, Xanthophyceae, and Euglenophyceae.

Plants for the production of desired sequences can be grown either in vivo and/or in vitro depending on the type of the selected plant and the geographic location. It is important that the selected plant is plant amenable to cultivation under the appropriate field conditions and/or in vitro conditions. The conditions for the growth of the plants are described in various basic books on botany, Agronomy, Taxonomy and Plant Tissue Culture, and are known to a skilled artisan in these fields.

Among angiosperms, the use of crop and/or crop-related members of the families identified in the paragraph below are particularly cotemplated. The plant members used in the present methods also include interspecific and/or intergeneric hybrids, mutagenized and/or genetically engineered plants. The term “crop member” refers specifically to species which are commercially grown as sources for vegetables, grains, forage, fodder, condiments and oilseeds. “Crop-related” members are those plants which have potential value as a crop and as donors of agronomically useful genes to crop members. Thus, crop-related members are able to exchange genetic material with crop members, thus permitting breeders and biotechnologists to perform interspecific (i.e., from one species to another) and intergeneric (i.e., from one genus to another) gene transfer. Those having ordinary skill in the art will understand that methods of exchanging genetic material between plants and testing effects of interspecific and intergeneric gene transfer are well characterized. See, for example Goodman et al., Science, 236: 48-54, 1987, incorporated herein by reference.

These families include and not limited to Leguminosae (Fabaceae) including pea, alfalfa, and soybean; Gramineae (Poaceae) including rice, corn, wheat; Solanaceae particularly of the genus Lycopersicon, particularly the species esculentum (tomato), the genus Solanum, particularly the species tuberosum (potato) and melongena (eggplant), the genus Capsicum, particularly the species annum (pepper), tobacco, and the like; Umbelliferae, particularly of the genera Daucus, particularly the species carota (carrot) and Apium, particularly the species graveolens dulce, (celery) and the like; Rutaceae, particularly of the genera Citrus (oranges) and the like; Compositae, particularly the genus Lactuca, and the species sativa (lettuce), and the like and the Family Cruciferae, particularly of the genera Brassica and Sinapis. Examples of “vegetative” crop members of the family Brassicaceae include, but are not limited to, digenomic tetraploids such as Brassica juncea (L.) Czem. (mustard), B. carinata Braun (ethopian mustard), and monogenomic diploids such as B. oleracea (L.) (cole crops), B. nigra (L.) Koch (black mustard), B. campestris (L.) (turnip rape) and Raphanus sativus (L.) (radish). Examples of “oil-seed” crop members of the family Brassicaceae include, but are not limited to, B. napus (L.) (rapeseed), B. campestris (L.), B. juncea (L.) Czem. and B. tournifortii and Sinapis alba (L.) (white mustard).

For example, it is known in the art that alfalfa mosaic virus has full host range. For example, species susceptible to virus: Abelmoschus esculentus, Ageratum conyzoides, Amaranthus caudatus, Amaranthus retrof lexus, Antirrhinum majus, Apium graveolens, Apium graveolens var. rapaceum, Arachis hypogaea, Astragalus glycyphyllos, Beta vulgaris, Brassica campestris ssp. rapa, Calendula officinalis, Capsicum annuum, Capsicum frutescens, Caryopteris incana, Catharanthus roseus, Celosia argentea, Cheiranthus cheiri, Chenopodium album, Chenopodium amaranticol, Chenopodium murale, Chenopodium quinoa, Cicer arietinum, Cichium endiva, Ciandrum sativum, Crotalaria spectabilis, Cucumis melo, Cucumis sativus, Cucurbita pepo, Cyamopsis tetragonoloba, Daucus carota (var. sativa), Dianthus barbatus, Dianthus caryophyllus, Emilia sagittata, Fagopyrum esculentum, Glycine max, Gomphrena globosa, Helianthus annuus, Lablab purpureus, Lactuca sativa, Lathyrus odatus, Lens culinaris, Linum usitatissimum, Lupinus albus, Lycopersicon esculentum, Macroptilium lathyroides, Malva parvifa, Matthiola incana, Medicago hispida, Medicago sativa, Melilotus albus, Nicotiana bigelovii, Nicotiana clevelandii, Nicotiana debneyi, Nicotiana glutinosa, Nicotiana megalosiphon, Nicotiana rustica, Nicotiana sylvestris, Nicotiana tabacum, Ocimum basilicum, Petunia x hybrida, Phaseolus lunatus, Phaseolus vulgaris, Philadelphus, Physalis flidana, Physalis peruviana, Phytolacca americana, Pisum sativum, Solanum demissum, Solanum melongena, Solanum nodiflum Solanum nodijlum, Solanum rostratum, Solanum tuberosum, Sonchus oleraceus, Spinacia oleracea, Stellaria media, Tetragonia tetragonioides, Trifolium dubium, Trifolium hybridum, Trifolium incarnatum, Trifolium pratense, Trifolium repens, Trifolium subterraneum, Tropaeolum majus, Viburnum opulus, Viciafaba, Vigna radiata, Vigna unguiculata, Vigna unguiculata ssp. sesquipedalis, and Zinnia elegans.

In sum, the plant members used in the present invention are plants that: (a) can be grown to high biomass in a short time either in vivo or in vitro; (b) are adaptable for growth in various agroclimatic conditions; (c) are adaptable to modified, non-conventional agricultural practices, described herein, for monoculture; (d) are amenable to genetic manipulation by mutagenesis and/or gene transfer; and (e) can produce several crops per year. Additionally the plant members are natural hosts for a selected virus. Alternatively, the selected virus can be made compatible with a plant so as to function as a host.

Depending on the type of host plant (lower plants to higher plants in the plant kingdom) used, infected or systemically infected host plant or tissue thereof can be harvested 10 days after inoculation, preferably 14 days after inoculation and more preferably 16 days after inoculation. Samples for the analysis (detection and quantification) of recombinant viruses and desired sequences can be taken from crude extracts of infected plant and from purified recombinant virus. Recombinant viruses can be purified from infected tissue can be easily accomplished using standard virus purification procedures known in the art.

Polypeptides and polynucleotides of interest can be recovered and purified from recombinant viruses by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. High performance liquid chromatography can be employed for purification. Well known techniques for refolding protein can be employed to regenerate active conformation when the polypeptide is denatured during isolation and or purification.

Purification techniques other than the affinity procedures outlined above can be used to purify, or supplement the purification of, a protein of the invention. Such methods can include without limitation, preparative electrophoresis, FPLC (Pharmacia, Uppsala, Sweden), HPLC (e.g., using gel filtration, reverse-phase or mildly hydrophobic columns), gel filtration, differential precipitation (for instance, “salting out” precipitations) and ion-exchange chromatography. The matrix used to create the affinity matrices will preferably comprise a carbohydrate matrix such as cross-linked dextran (e.g., that sold under the tradename Sepharose) or agarose (e.g., that sold by Pharmacia, Sweden as “Sephacryl”). The matrix should have pore sizes sufficient to admit both the affinity ligand that will be attached to the matrix and the multifunctional enzyme of the invention. Methods of synthesizing appropriate affinity columns are well known. See, for instance, Axèn et al., Nature, 214:1302.1304, 1967.

The polypeptides and nucleic acids in the recombinant viruses are detected and quantified by any of a number of means well known to those of skill in the art. The infected plants can show symptoms specific to each virus. Such symptom production can be a useful detection marker. A number of laboratory techniques can also reliably be employed for the detection. These include analytic biochemical methods such as spectrophotometry, radiography, electrophoresis, capillary electrophoresis, high performance liquid chromatography (HPLC), thin layer chromatography (TLC), hyperdiffusion chromatography, and the like, and various immunological methods such as fluid or gel precipitin reactions, immunodiffusion (single or double), immunoelectrophoresis, radioimmunoassays (RIAs), enzyme-linked immunosorbent assays (ELISAs), immunofluorescent assays, and the like. The detection of nucleic acids proceeds by well known methods such as northern analysis, gel electrophoresis, PCR, radiolabeling and scintillation counting, and affinity chromatography.

One of skill will appreciate that it is often desirable to reduce non specific binding in immunoassays and during analyte purification. Where the assay involves a polypeptide, antibody, or other capture agent immobilized on a solid substrate, it is desirable to minimize the amount of non specific binding to the substrate. Means of reducing such non specific binding are well known to those of skill in the art. Typically, this involves coating the substrate with a proteinaceous composition. In particular, protein compositions such as bovine serum albumin (BSA), nonfat powdered milk, and gelatin are widely used.

Western blot analysis can also be used to detect and quantify the presence of a transcript polypeptide or antibody or enzymatic digestion product) in the sample. The technique generally comprises separating sample products by gel electrophoresis on the basis of molecular weight, transferring the separated proteins to a suitable solid support, (such as a nitrocellulose filter, a nylon filter, or derivatized nylon filter), and incubating the sample with labeling antibodies that specifically bind to the analyte protein. The labeling antibodies specifically bind to analyte on the solid support. These antibodies are directly labeled, or alternatively are subsequently detected using labeling agents such as antibodies that specifically bind to the labeling antibody. To prevent nonspecific binding of compound (e.g., labeled antibody) to the surface of the solid support, the surface is typically blocked with a second compound (e.g., milk).

Labeling agents include e.g., monoclonal antibodies, polyclonal antibodies, proteins such as those described herein, or other polymers such as affinity matrices, carbohydrates or lipids. Detection proceeds by any known method, such as immunoblotting, western analysis, gel-mobility shift assays, fluorescent in situ hybridization analysis (FISH), tracking of bioluminescent markers, nuclear magnetic resonance, or other methods which track a molecule based upon size, charge or affinity. Thus, a label is any composition detectable by spectroscopic, photochemical, biochemical, immunochemical, electrical, optical or chemical means. Useful labels in the present invention include magnetic beads (e.g. Dynabeads.™.), fluorescent dyes (e.g., fluorescein isothiocyanate, rhodamine, and the like), radiolabels (e.g., ³H, ²⁵I, ³⁵S, ¹⁴C, or 32P), and nucleic acid intercalators (e.g., ethidium bromide)

The label is coupled directly or indirectly to the desired component of the assay according to methods well known in the art. As indicated above, a wide variety of labels are used, with the choice of label depending on the sensitivity required, ease of conjugation of the compound, stability requirements, available instrumentation, and disposal provisions.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence, e.g., by fluorescence microscopy, visual inspection, via photographic film or by the use of electronic detectors and the like.

Animal or human hosts infected by a pathogen or vaccine antigens (or antigenic or immunogenic determinants) mount an immune response in response to the invading pathogen or the vaccine antigen. The immune system works in three fundamentally different ways which is well known in the art. See, Lodish et al., MOLECULAR CELL BIOLOGY, Scientific American Books, New York (1995); Roitt et al. IMMUNOLOGY, Mosby International, London (1998) which are incorporated herein by reference. Accordingly, The foreign polypeptides or polynucleotides or cells expressing them produced according to the methods described herein can be used as an antigen or as an immunogen for vaccination of an animal including human to produce specific antibodies which have anti-bacterial anti-viral and/or anti-cancerous action. In addition, polypeptides in which one or more of the amino acid residues are modified (i.e., derivatives of polypeptides) can also be used. Such polypeptides, for example, can be the result of substitution, addition, or rearrangement of amino acids or chemical modification thereof. All such substitutions and modifications are generally well known to those skilled in the art of peptide chemistry.

This invention also contemplates the use of the foreign nucleic acids encoding the antigen as a component in a DNA vaccine as discussed further below.

Another aspect of the invention relates to a method for inducing an immunological response in an animal, particularly a human which involves administering the animal an effective amount of plant cells or tissue containing a vaccine antigen, or a purified vaccine antigen produced according to the method herein adequate to produce antibody and/or T cell immune response to protect said animal from infection and/or disease caused by pathogens. Also provided are methods whereby such immunological response slows bacterial or viral replication or a parasitic pathogen in the animal whether that disease is already established within the animal or not.

Polypeptides and their derivatives include immunologically equivalent derivatives which form a particular aspect of this invention. The term ‘immunologically equivalent derivative’ as used herein encompasses a peptide or its equivalent which when used in a suitable formulation to raise an immunological response in an animal which response acts to interfere with the interaction between pathogen and mammalian host. The immunological response may be used therapeutically or prophylactically and may take the form of antibody immunity or cellular immunity such as that arising from CTL or CD4+ T cells.

The polypeptide, such as an immunologically equivalent derivative or a fusion protein thereof is used as an antigen to immunize the animal (see Example 7). The fusion protein can provide stability to the polypeptide. The antigen may be associated, for example by conjugation, with an immunogenic carrier protein for example, protective antigen of authrax bacteria and heat shock proteins which can facilitate the transport of administered antigen to cell cytosol. Alternatively, a multiple antigenic peptide comprising multiple copies of the protein or polypeptide, or an immunologically equivalent polypeptide thereof may be sufficiently antigenic to improve immunogenicity so as to obviate the use of a carrier.

The invention also includes a vaccine formulation which comprises an immunogenic recombinant protein of the invention together with a suitable carrier. Since the protein may be broken down in the stomach, it is preferably administered parenterally, including, for example, administration that is subcutaneous, intramuscular, intravenous, or intradermal. Formulations suitable for parenteral administration include aqueous and non-aqueous sterile injection solutions which may contain anti-oxidants, buffers, bacteriostats and solutes which render the formulation isotonic with the bodily fluid, preferably the blood, of the individual; and aqueous and non-aqueous sterile suspensions which may include suspending agents or thickening agents. The formulations may be presented in unit-dose or multi-dose containers, for example, sealed ampules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. In therapy or as a prophylactic, the active agent i.e., the desired vaccine antigen (polypeptide or polynucleotide) can be administered to a patient as an injectable composition, for example as a sterile aqueous dispersion, preferably isotonic. The dosage will depend on the specific activity of the vaccine and can be readily determined by routine experimentation.

As noted earlier, the plant viruses replicate and express at a high rate in plant cells, thereby leading to the rapid production of large numbers of virus particles with the attached foreign sequences following infection. Many viruses have between 10⁶ to 10⁷ particles per cell.

Thus, there is on the order of 10-100 pg of viral coat protein per cell. Thus, according to the methods of the present invention using the recombinant viral vectors, foreign polypeptides in the order of 0.5-1.0 mg can be produced per gram of plant tissue when the foreign polypeptide is fused to the coat protein of a given virus.

The methods of producing fused coat proteins with antigenic or non-antigenic foreign polypeptides using plant viruses and methods of delivering a fused coat protein to an animal for purposes of inducing an immune response against the foreign polypeptide has been demonstrated in WO 98/08375, the contents of which are incorporated herein by reference.

The following examples further illustrate the present invention, but of course, should not be construed as in any way limiting its scope. The examples below are carried out using standard techniques, that are well known and routine to those of skill in the art, except where otherwise described in detail. The examples are illustrative, but do not limit the invention. All animal methods of treatment or prevention described herein are preferably applied to mammals, most preferably humans.

### EXAMPLE 1

### Construction of NF1 to Express Different Foreign Peptides

The starting plasmid pCPΔAUG (Loesh-Fries et. al, 1985, Virology 146, 177-187) contains an AlMV coat protein modified so that the AUG translation initiation codon is replaced by TCG to create an Xhol (CTCGAG) site for cloning and an RNA molecule defective in translation. pSPΔAUG was used to create all NF1 constructs (FIGS. 1A and B).

Shown in FIG. 1 is a schematic representation of cloning of foreign peptides as a translational fusions of AlMV CP in full-length RNA3 of AlMV. (A) represents cloning strategy used. Two ellipsoids represent a foreign peptide with XhoI and Sal I cloning sites. CPΔAUG is AlMV CP where the translation initiation codon (AUG) is mutated to create XhoI cloning site. T7P3Sal is 5′ portion of AlMV RNA3 containing ORF for P3, 5′ non coding regions of RNA3 and subgenomic RNA4. The Sal I site is created at the position 1192 to mutate the AlMV CP AUG. Additionally T7P3Sal contains T7 promoter for in vitro synthesis of infectious RNA3 transcripts. NF 1 is full-length RNA3 containing foreign peptide fused to AlMV CP. (B) pNF1/g24 is RNA3 containing epitope from rabies glycoprotein. pNF1/RSV contains 24 amino acid epitope from RSV G protein. pNF1/Sand contains octerotide sandostatin and pNF1/TVE contains CP fused with 104 amino acids from colorectal cancer GA733-2. Amino acid sequences of cloned peptides are shown under each construct. Stem-loop structure indicates the 3′ non coding region of RNA3.

It should be noted that only those constructs that have the foreign polypeptide-encoding nucleic acid ligated in the right orientation is used. Right-orientation of the foreign polypeptide-encoding nucleic acid is with the 5¹-end thereof proximal to the promoter. Right orientation is confirmed by restriction digest analysis or by sequencing analysis.

a. pNF1/RSV (FIG. 1B) PCR cloning was used to create a fusion protein consisting of a full-length AlMV coat protein and the 24 amino acid peptide from the G protein of the Respiratory Syncytial Virus (RSV), shown to protect (Bastien et. al., 1997, Virology 234,118-22) immunized mice against infection with RSV. DNA sequences encoding these 24 amino acids (FIG. 1B; SEQ ID NO: 1) were PCR amplified and cloned into the AlMV genome by translational fusion with the coat protein. The epitope was amplified using 5′GCGCTCGAGCATCATGTCACCCTGCAGCATATGCAGCAACAATCCA3′ (SEQ ID NO: 2) as a first strand primer and 5′CGCGTCGACTTGCAGATAGCCCAGCAGGTTGGATTGTTGCT3′ (SEQ ID NO: 3) as a second strand primer. A 90-basepair (bp) PCR fragment (encoding 24 amino acids) containing the RSV epitope with introduced XhoI and SalI sites at 5′- and 3′-ends respectively, was digested with Xhol+SalI and ligated into pSPΔAUG linearized by Xhol to engineer CPRSV. The resulting plasmid is termed pSPCPRSV. Translation of the recombinant protein initiates from the AUG codon created at the 5′ end of the chimeric gene, which is upstream of nucleotide sequences encoding the RSV epitope. The recombinant plasmid also contains linking 5′-(37 nucleotides upstream from the wild-type AlMV coat protein translation start codon) and 3′-(192 nucleotides following the AlMV coat protein stop codon and containing the AlMV origin of assembly) noncoding regions of the AlMV coat protein. The epitope was fused to the N-terminus of the coat protein. After sequence confirmation, the recombinant coat protein was subcloned into full-length RNA3 of AlMV to create pNF1RSV (FIG. 1B). Using the full-length coat protein and the strategy described herein, additional constructs were engineered as follows:

b. pNF1/g24 (FIG. 1B). PCR was performed using 5′GCGCTCGAGGGTACCATGTCCGCCGTCTACACCCGAATTATGATGAACG GAGGACGACTTAAGCGACCACCAGACCAGCTTG3′ (SEQ ID NO: 4) as a first strand primer and 5′CGAGGTACCCTCTTCCACCACAAGGTGCTCATTTTCGTCGGATCGGAAGT CGTGAAGGTTCACAAGCTGGTCTGGTGGTCGCTTAAGTCGTCC3′(SEQ ID NO: 5) as a second strand primer. NF I Drg24 contains the coat protein fused with a rabies epitope capable of protecting the immunized mice against a lethal dose of challenge rabies virus. The next step involved the engineering of a linear epitope, G5-24 of rabies virus glycoprotein as a chimera (Drg24) with an epitope from the rabies virus nucleoprotein (31D). This chimera was fused with the AlMV coat protein. The chimeric epitope, Drg24, was synthesized by PCR using oligonucleotides containing 18 complementary nucleotides between the first and second, such that the complementary nucleotide strands can anneal and initiate the PCR reaction. The 120 (coding for 40 amino acids; SEQ ID NO: 6) PCR product was digested with XhoI and cloned into pSPDAUG to create pSPCPDrg24. The latter was combined by ligation with the 5′ part of AlMV RNA3 to obtain pNF1/g24.

c. pNF1/Sand (FIG. 1B). Sandostatin is an 9 amino acid peptide (SEQ ID NO: 7) used to suppress the synthesis of human growth hormone in diseased people. Sandostatin was fused with the coat protein of AlMV by PCR using 5′GCGGAATTCGTTTTTATTTTTAATTTTCTTTCAATTACTTCCATCATGAGT TCTTTCTGTTTCTGGAAA3′ (SEQ ID NO: 8) as a first strand primer and 5′GCGCTCGAGCGAGTACACGTTTTCCAGAAACAGAA3′ (SEQ ID NO: 9) as a second strand primer. The fusion product was then cloned into full-length RNA3 as described above.

d. pNFI/TVE (FIG. 1B). 104 amino acid peptides from colorectal cancer antigen GA733-2 (Linnenbach et al., 1989, Proc. Natl. Acad. Sci. USA 86: 27-31) were fused with mutant AlMV CP. This region has a special conformation recognized by colorectal cancer associated antibody 17-1A. The sequences encoding this region (104 amino acids; SEQ ID NO: 10) of GA733-2 were PCR amplified using 5′GCGCTCGAGGGTACCATGCGACGGCGACTTTTGCCGCA3′ (SEQ ID NO: 11) as a first strand primer and 5′GTCGACCTGGTACCAGTGTTCACACACCAGCACG3′ (SEQ ID NO: 12) as a second strand primer. The PCR products were digested by XhoI+ SalI and cloned into pSPDAUG by XhoI. The resulting plasmid is pNF1/TVE (FIG. 1B).

### EXAMPLE 2

### Construction of NF2 to Express Different Foreign Peptides

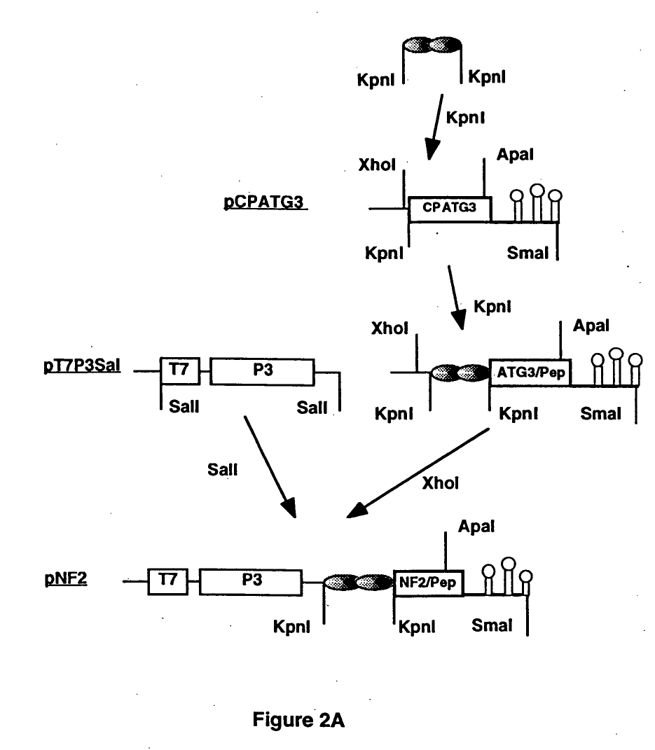

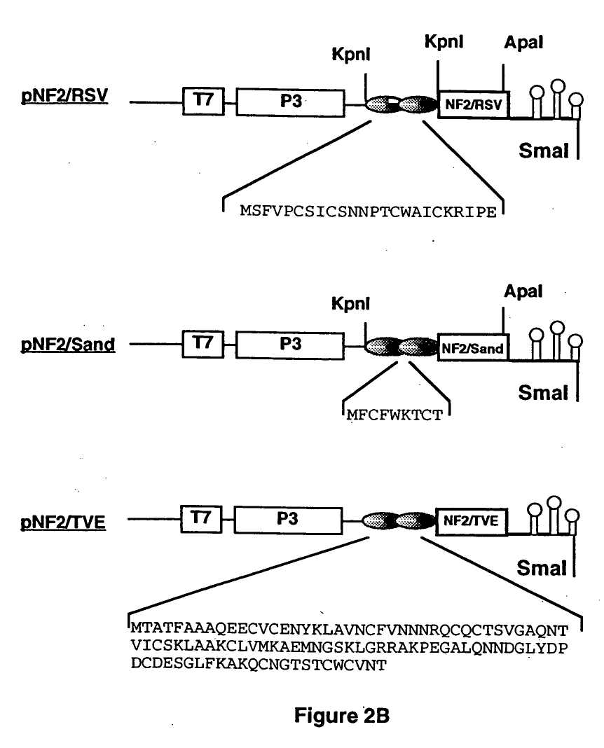

Assembly of fusion proteins into virus particles greatly enhances the ability to purify the peptides or polypeptides from plants simply by the isolation of the virus (see Example 3). Some of the products (peptides), however, may require separation from fusion protein (particles) for functional activity. The AlMV coat protein has a natural trypsin recognition site, which allows cleavage between amino acid 24 and 25 at the N-terminus of the coat protein. When the peptides are fused to NF1, the cleavage with trypsin will result in a foreign peptide carrying 24 N-terminal amino acids of AlMV CP at their C-terminus. This can be detrimental for the functional activity of some peptides. To improve this feature of the carrier molecule, a number of AlMV coat protein mutants were tested. The deletion of 12 N-terminal, amino acids of AlMV coat protein resulted in a mutant molecule, which retains the function of the wild type and is capable of accommodating the same size peptides as the wild type. The new clone, pSPDATG3 contains all the wild type RNA sequences. It is derived from pSP65DAUG by creating a KpnI cloning site at position 1226 (RNA3 sequence), which is 33 nucleotides (11 amino acids) downstream from the original translation initiation site (AUG). The KpnI site at position 1226 was introduced by PCR using 5′GCGCTCGAGTTCTTCACAAAAGAAAGCTGGTGGGAAAAGGTACCGCTGG TAAACCT3′ (SEQ ID NO: 13) as a first strand primer and 5′ATTAAAAGAGCTCAGACTC3′ (SEQ ID NO: 14) as second strand primers. The PCR product was digested by Xhol+SstI and cloned into pSP65ΔAUG cleaved by Xhol+Sstl to replace the original DNA fragment with a mutant one (FIG. 2).

Illustrated in FIG. 2 is a schematic representation of cloning of foreign peptides as a translational fusions with mutant AlMV CP (CPATG3) in full-length RNA3 of AlMV. (A) represents cloning strategy used. Two ellipsoids represent a foreign peptide with KpnI cloning site. CPATG3 is a mutant AlMV CP where the translation initiation codon (AUG) is mutated to create Xho I site and KpnI at position 1226 for the cloning of foreign peptides. T7P3Sal is 5′ portion of AlMV RNA3 containing ORF for P3, 5′ non coding regions of RNA3 and subgenomic RNA4. The Sal I site is created at the position 1192 to mutate the AlMV CP AUG and to clone into Xho I site at the same position in CPATG3. Additionally T7P3Sal contains T7 promoter for in vitro synthesis of infectious RNA3 transcripts. NF2 is full-length RNA3 containing foreign peptide fused to AIMV CP. (B) pNF2/RSV contains 24 amino acid epitope from RSV G protein. pNF2/Sand contains octerotide sandostatin and pNF2/TVE contains CP fused with 104 amino acids from colorectal cancer GA733-2. Amino acid sequences of cloned peptides are shown under each construct. Stem-loop structure indicates the 3′ non coding region of RNA3.

To compare the mutant and wild type protein we engineered the fusions with the following peptides:

a. pNF2/RSV (FIG. 2B). DNA sequence coding for antigenic epitope of RSV G protein was PCR amplified using 5′GCGCTCGAGGGTACCATGTCCTTTGTACCCTGCAGCATATGCAGCAACAATCCA3′ (SEQ ID NO: 15) as a first strand and 5′CGAGGTACCCTCTGGTATTCTTTTGCAGATAGCCCAGCAGGTTGGATTGTTGCT3′ (SEQ ID NO: 16) as second strand primers. During PCR the KpnI site was introduced for cloning into NF2. The PCR product was digested by KpnI and ligated into NF2 linearized by KpnI to obtain NF2RSV. pNF2/RSV consists of full-length RNA3 where the antigenic epitope of RSV G protein is fused to the N-terminus of mutant coat protein CPDATG3.

b. pNF2/Sand (FIG. 2B). The sequences encoding sandostatin were PCR amplified using 5′GCGGGTACCATGTTCTGTTTCTGGAAAACGTGTACTGCTGGTAAACCTAC TAAACGT3′ (SEQ ID NO: 17) as a first strand and 5′GCGCTCGAGCATCCCTTAGGGGCATTCATGCA3′ (SEQ ID NO: 18) as second strand primers. The first strand primer contains sequences for both sandostatin (27 nucleotides) and AlMV CP (19 3′ nucleotides) for annealing. Thus, the PCR product will contain sequences encoding sandostatin and all the sequences of AlMV CP downstream of the nucleotide 1226 of RNA3. Therefore, the PCR product was digested by KpnI (newly introduced) Apal (in original AlMV sequences), where the sequences encoding sandostatin were followed by the sequences encoding mutant coat protein CPDATG3 and were cloned into NF2RSV by KpnI Apal to replace an identical region of coat protein together with the fused RSV epitope. The resulting plasmid is pNF2/Sand (FIG. 2B).

c. pNF2/TVE (FIG. 2B). 104 amino acid peptides from colorectal cancer antigen GA733-2 (Linnenbach et al., 1989, Proc. Natl. Acad. Sci. USA 86: 27-31) were fused with mutant AlMV CP. This region has special conformation recognized by colorectal cancer associated antibody 17-1A. The sequences encoding this region (80 amino acids) of GA733-2 were PCR amplified using 5′GCGCTCGAGGGTACCATGCG ACGGCGACTTTTGCCGCA3′ (SEQ ID NO: 19) as a first strand and 5′GTCGACCTGGTACCAGTGTT CACACACCAGCACG3′ (SEQ ID NO: 20) as second strand primers. The PCR products were digested by KpnI and cloned into NF2RSV by Kpn I to replace the RSV epitope fused with the mutant coat protein. The resulting plasmid is pNF2/TVE (FIG. 2B).

### EXAMPLE 3

### Construction of RNA3a and RNAM to Produce Full-Length Protein

AlMV has three genomic RNAs. RNA1 and 2 encode for P1 and P2 proteins required for the replication of viral RNA. RNA3 encodes for P3 (cell to cell movement) and coat protein (long distance movement and encapcidation). Coat protein is translated from subgenomic RNA4. RNA4 is synthesised from genomic RNA3. P3 and coat protein are required for virus to be fully infectious. Deletion of either of these two proteins will limit the infectivity of viruses. Based on the importance of these two proteins for virus infectivity we introduced mutations into RNA3 to create two new molecules (RNA3a and RNA3b, FIG. 2). RNA3a has functionally active P3 and is deficient in coat protein production. RNA3b has functionally active coat protein and is deficient in P3 production. The functions of coat protein and P3 can be complemented from two different molecules (RNA3a and 25 RNA3b) replacing the wild type RNA3. The RNA3a is NF2 (see Example 2) where the wild type coat protein is replaced with mutant coat protein. The mutations were introduced to eliminate the AlMV CP translation initiation codon and to create KpnI site for subcloning at position 1226. Thus, there is no translation initiation codon for coat protein gene in RNA3a. Instead it has XhoI (position 1192 of RNA3), Kpnl and Apal (position 1800 of RNA3) cloning sites for replacing the coat protein sequences with the sequences of the desired gene using Xhol-Apal or KpnI-Apal (FIG. 2).

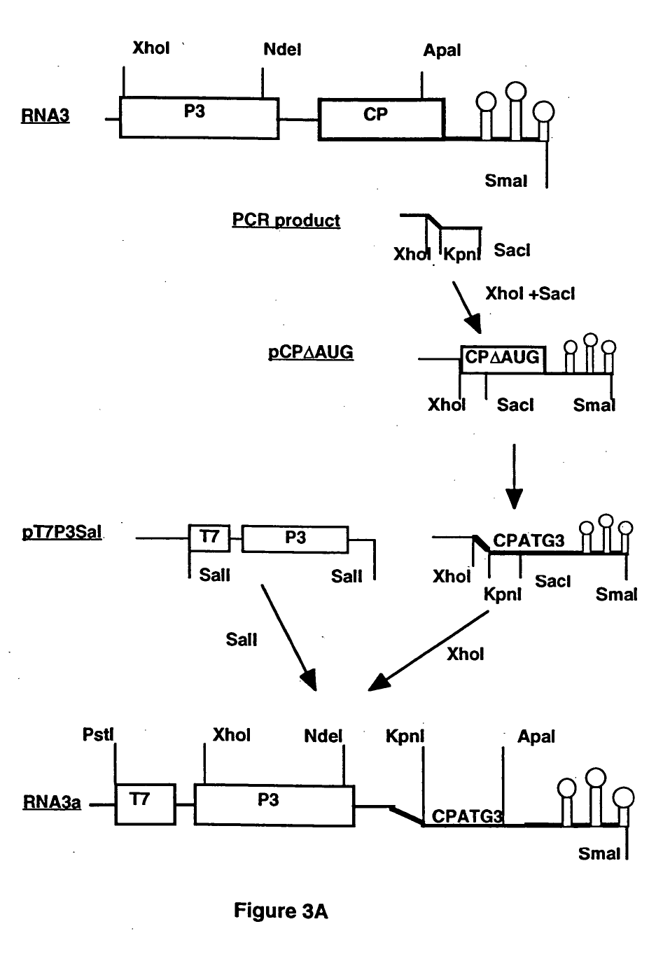

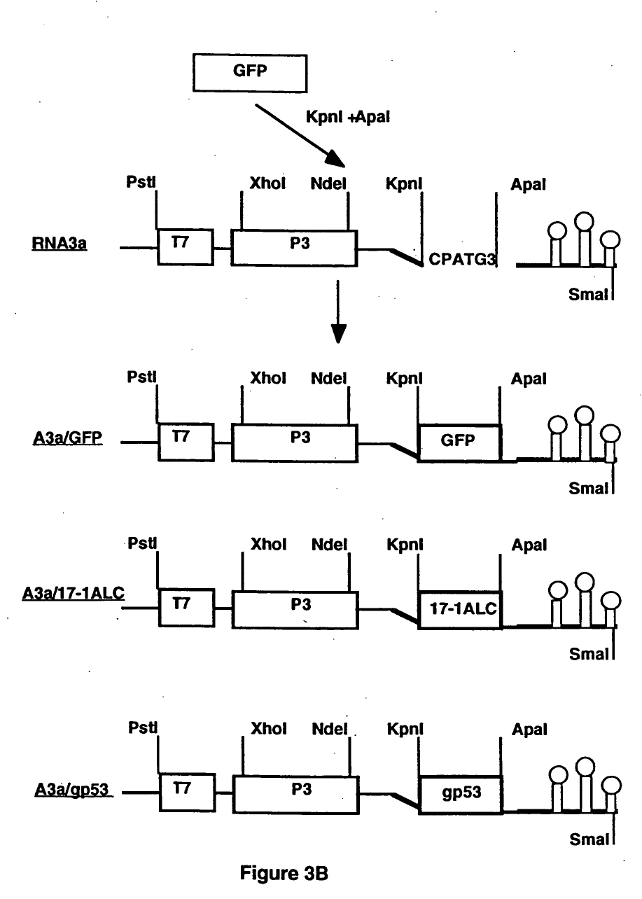

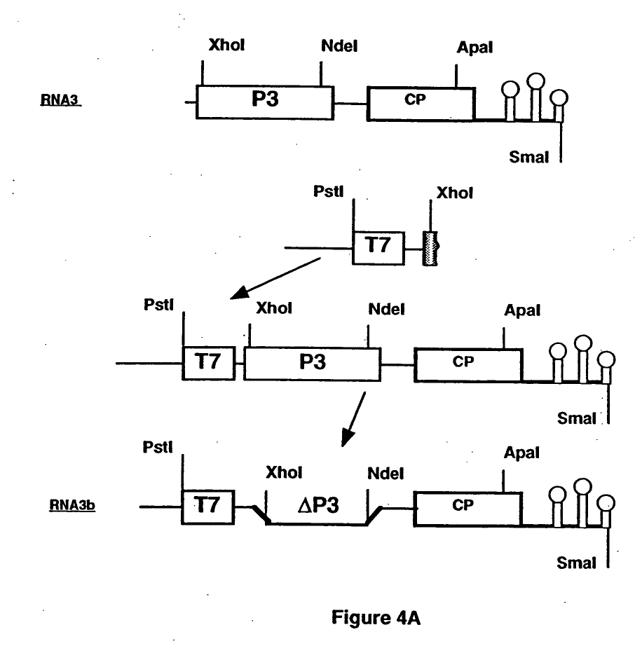

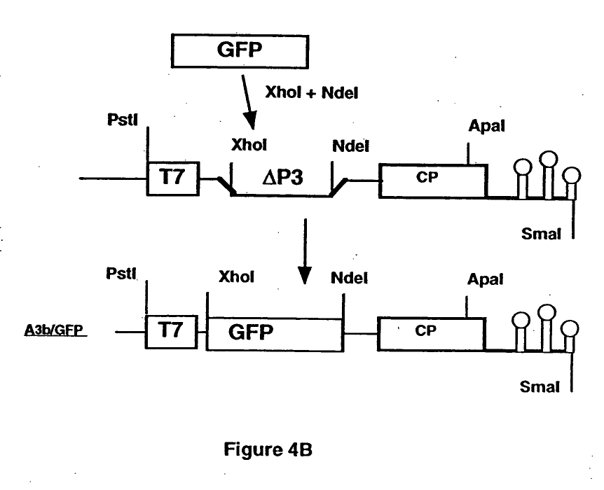

In RNA3b we replaced the translation initiation codon for P3 (ATG) with Nhel restriction site by PCR using 5′GCACTCATTCAACATTGCTAGCTTATGTTTTTGTTTACGGAGCTCAAG3′ (SEQ ID NO: 21) as a second strand primer and 5′CATGCCATTGAMAGGTGACACAATAG3′ (SEQ ID NO: 22) as a firststrand primer. As a template we used T7RNA3. The amplified DNA fragment containing T7 promoter and 5245 nucleotides of RNA3 with mutation was digested by PstI XhoI and cloned into T7RNA3 cleaved by PstI XhoI to replace the wild type sequences. Thus, RNA3b has XhoI and NdeI restriction sites (FIG. 4) for replacing the open reading frame of P3 with the sequences of the desired gene. This system allows expression of 1 or 2 genes simultaneously. Using this strategy we cloned the following proteins into RNA3a or RNA3b (FIG. 3A or 4A):

Shown in FIG. 3 is the construction of RNA3a. RNA3 is wild-type genomic RNA of AlMV. The boxes indicate the ORF's for movement protein P3 and for coat protein CP. Stem-loop structure indicates the 3′ non coding region of RNA3. (A) The PCR product containing newly introduced KpnI site and mutated AUG codon is cloned into CPDAUG to create CPATG3. CPATG3 is deficient in translation of AlMV CP and has KpnI cloning site at position 1226. Then the CPATG3 is combined with T7P3Sal using Xho I and Sal I sites for cloning as described in FIG. 2 to obtain RNA3a. (B) A3a/GFP—the ORF for AlMV CP is replaced with that of GFP. A3a/17-1-ALC contains the ORF for LC of colorectal cancer associated antibody 171A. A3a/gp53 contains the glycoprotein from bovine viral diarrhoea virus.

Construction of RNA3b is shown in FIG. 4 RNA3 is wild-type genomic RNA of AIMV. The boxes indicate the ORF's for movement protein P3 and for coat protein CP. Stem-loop structure indicates the 3′ non coding region of RNA3. (A) The PCR product containing T7 promoter and mutated AUG codon of P3 is cloned into T7/A3 the infectious cDNA clone of AlMV RNA3 using Pst I+Xho I restriction sites. The resulting plasmid is RNA3b. RNA3b is deficient in translation of P3 and has Xho I (position 245) NdeI (positions 1082) cloning sites. (B) shows A3b/GFP—in which the ORF for P3 is replaced with that of GFP. Stem-loop structure indicates the 3′ non coding region of RNA3.

a. GFP (green fluorescent protein) from jellyfish (FIG. 3B) GFP has been used as a marker for expression in different systems. We amplified GFP to introduce Kpnl and Apal restriction sites for cloning using 5′GCGGGTACCGTCOACGOCCAOATCGOCCATGAGTAAAOGAGAAGAAC3′ (SEQ ID NO: 23) as a first strand and 5′GCGGGCCCATTAATGCGGCCGCTCATTTGTATAGTTCATCC3′ (SEQ ID NO: 24) as second strand primers. The amplified PCR product was digested by Kpnl+Apal and cloned into pNF2/RSV cleaved by Kpnl+Apal. The resulting clone pA3a/GFP contains 5′- and 3′-non coding regions of RNA3 and ORF's for P3 and GFP. The open reading frame of the coat protein between nucleotides 1226-1800 was replaced with an open reading frame of GFP.

b. gp53 of Bovine Viral Diarrhea Virus (BVDV, FIG. 3B). gp53 is shown to stimulate virus neutralizing antibodies in vitro and in vivo. gp53 is a major component of diagnostic kits as well as vaccine preparations used to detect and prevent BVDV. We cloned the open reading frame of gp53 from NADI strain of BVDV by RT-PCR into pGEM-T (Promega, Madison, Wis.) expression vector using 5′GCGGGCCCATTAATGCGGCCGCTCATTTGTATAGTTCATCC3′ (SEQ ID NO: 25) as a second strand and 5′GCACTCGAGTTACTCACTTGATATGATTTCATATGGTCT3′ (SEQ ID NO: 26) as first strand primers. After sequence confirmation, the ORF of gp53 was cleaved by Pacl+Xhol and cloned into pNF2/RSV digested by KpnI+ApaI using blunt end ligation to create pA3a/gp53 (FIG. 3B).

c. Light chain (LC) of monoclonal antibody 17-1A (FIG. 3B). 17-1A is the monoclonal antibody (Linnenbach et al., 1989, Proc. Natl. Acad. Sci. USA 86: 27-31) against colorectal cancer associated with GA733-2 antigen. The gene encoding light chain of 17-1A was amplified using GCGTTAATTAAGGCCAGATCGGCCATGGGCATCAAGATGGGATCA3′ (SEQ ID NO:-27) as a first strand and 5′GCGTTAATTAAGCGGCCGCCTAACACTCATTCCTGTTGAA3′ (SEQ ID NO: 28) as a second-strand primer and cloned into pGEM-T vector. After sequence confirmation the DNA fragment containing ORF of 17-1A LC was cleaved by Pad and cloned into pNF2/RSV digested by KpnI+ApaI using blunt end ligation to create pA3a/17-1ALC (FIG. 3B).

d. GFP was also cloned into RNA3b to replace the ORF for P3 and to create pA3b/GFP (FIG. 4B). The ORF of GFP was cleaved from pGEM-GFP by KpnI+Apal and cloned into RNA3b cleaved by NheI+NdeI to replace the ORF of P3. The resulting clone pA3b/GFP (FIG. 4B) contains 5′- and 3′-non coding regions of RNA3, AlMV CP and the GFP ORF. The open reading frame of P3 between nucleotides 245-1100 was replaced with an open reading frame of GFP.

### EXAMPLE 4

### Production of Foreign Peptides Fused to AlMV CP using Transgenic Plants Expressing the Replicase Proteins of AlMV

Rep plants are transgenic tobacco plants expressing replicase proteins (PI and P2) of AlMV. It has been demonstrated that inoculation of these plants with RNA3 only results in virus infection and systemic movement of RNA3. This shows that P1 and P2 expressed in transgenic plants will complement for virus replicase function.

To test our hypothesis of producing foreign peptides fused to AlMV coat protein using transgenic Rep plants, we inoculated the plants with in vitro transcripts of pNF1/RSV, pNF1/Sand, Pnf1/g24, pNF1/TVE, pNF2/RSV, pNF2/Sand and pNF2/TVE. Tobacco leaves were inoculated with in vitro transcription products of recombinant RNA3. The transcription products of recombinant RNA3 were diluted 1:1 in 30 mM sodium phosphate, pH 7.2, and applied onto expanding tobacco leaves after abrading the leaf surface with carborundurm (320 grit; Fisher, Pittsburgh, Pa.). Inoculation was affected by gentle rubbing to spread the inoculum and further abrade the leaf surface. The recombinant virus was isolated 12-14 days after the inoculum was applied, as described (Yusibov et al., 1997, Proc. Natl. Acad. Sci. USA 94, 5784-5788). Briefly, leaf tissue was ground and the sap separated from cell debris by centrifugation. Virus particles were selectively precipitated using 5% polyethylene glycol. Then the purified virus was analyzed for the presence of full-length recombinant protein and the peptide of interest using Western analysis.

Western Blot Analysis: Recombinant proteins produced in virus-infected plants were analyzed by Western blot (Yusibov et. al., 1997, Proc. Natl. Acad Sci. USA 94, 5784-5788). Proteins from purified virus particles were separated electrophoretically on SDS-polyacrylamide gels and electroblotted onto a nylon membrane overnight at 33 mA. After blocking with milk (Kirkegaard & Perry; Gaithersburg, Md.), proteins were allowed to react with appropriate antibodies and detected using a Vectastain ABC kit (Vector Laboratories, Burlingame, Calif.). The recombinant proteins were detected using antibodies specific for: the AlMV coat protein (Agdia, Elkhart, Ind.); the linear epitope (G5-24) of rabies glycoprotein (Dietzschold et. al., 1990, J Virology 64, 3804-3809).

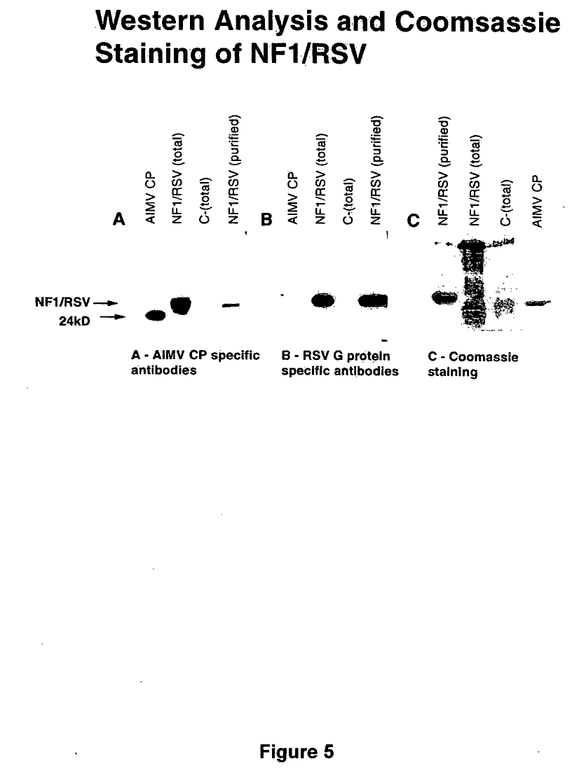

Shown in FIG. 5 is Western analysis and Coomassie staining of NF1/RSV. Proteins were separated by electrophoresis through a 13% SDS-polyacrylamide gel and bound with monoclonal antibodies specific for the AIMV coat protein (A), for the epitope of RSV G protein and stained with Coomassie (C). Wild type AIMV coat protein (24 kD) bound only with antibodies against AlMV coat protein (A) and did not bind with antibodies against fusion peptide (B). The fusion protein NF1/RSV, however, was recognized (A and B) with antibodies specific for both carrier molecule (AlMV CP) and fused peptide (RSV) in total extracts from infected leaves as well in purified virus samples. Total extracts (C-(total)) from noninoculated plants did not react with either of the antibodies. Coomassie staining of NF1/RSV in total extracts and in purified virus samples demonstrates the efficacy of purification procedure.

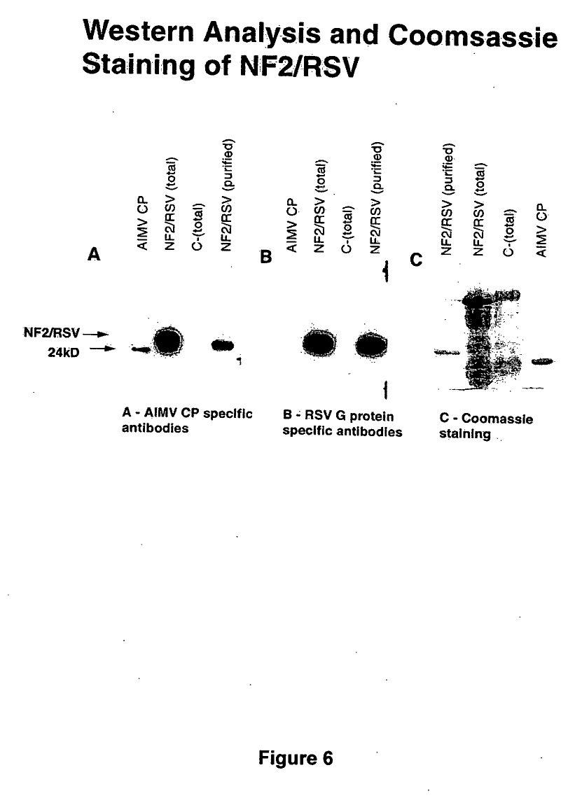

Shown in FIG. 6 is Western analysis and Coomassie staining of NF2/RSV. Proteins were separated by electrophoresis through a 13% SDS-polyacrylamide gel and bound with monoclonal antibodies specific for the AIMV coat protein (A), for the epitope of RSV G protein and stained with Coomassie (C). Wild type AIMV coat protein (24 kD) bound only with antibodies against AIMV coat protein (A) and did not bind with antibodies against fusion peptide (B). The fusion protein NF2/RSV, however, was recognized (A and B) with antibodies specific for both carrier molecule (AlMV CP) and fused peptide (RSV) in total extracts from infected leaves as well as in purified virus samples. Total extracts (C-(total)) from noninoculated plants did not react with either of the antibodies.

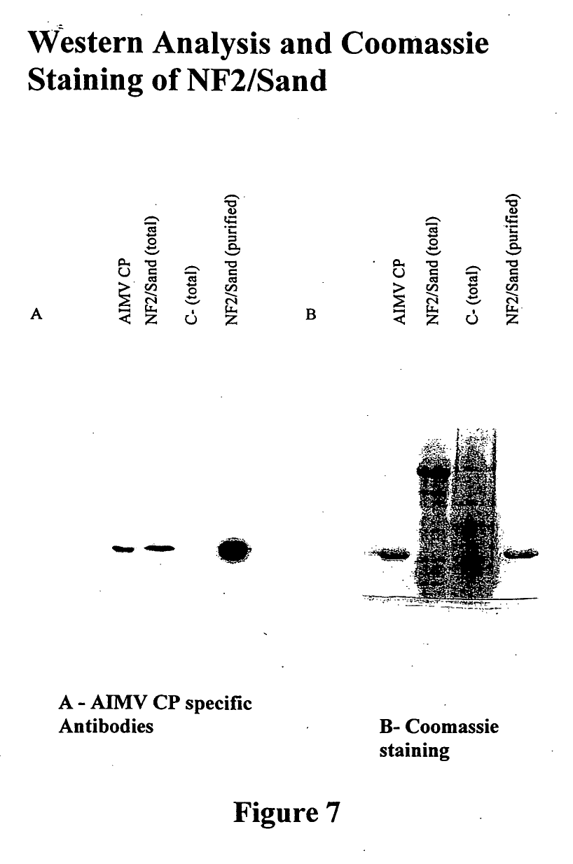

Shown in FIG. 7 is Western analysis and Coomassie staining of NF2/Sand. Proteins were separated by electrophoresis through a 13% SDS-polyacrylamide gel and bound with monoclonal antibody specific for the AlMV coat protein (A) and stained with Coomassie (B). The antibody reacted with AlMV coat protein (24 kD) and with fusion protein NF2/Sand (A). The NF2/Sand was recognized (A and B) with antibody in total extracts from infected leaves as well in purified virus samples. Total extracts (C-(total)) from noninoculated plants did not react with either of the antibodies. Coomassie staining of NF2/Sand in total extracts and in purified virus samples demonstrates the efficacy of purification procedure.

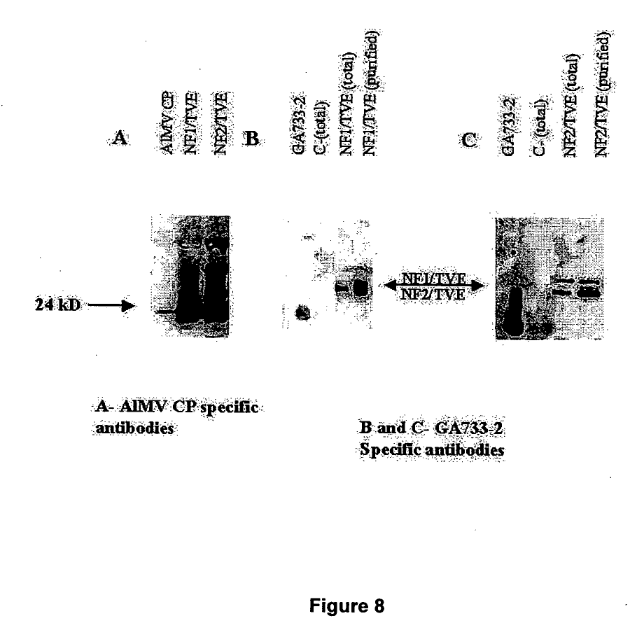

Shown in FIG. 8 is Western analysis of NF1/TVE and NF2/TVE accumulation and assembly in infected plants. Proteins were separated by electrophoresis through a 13% SDS-polyacrylamide gel and bound with monoclonal antibodies specific for the AIMV coat protein (A) and for colorectal cancer antigen GA733-2 (B-NF1/TVE and C-NF2/TVE). Wild type AlMV coat protein (24 kD) bound only with antibodies against AIMV coat protein (A) and did not bind with antibodies against fusion peptide (B and C). The fusion proteins NF1/TVE and NF2/TVE, however, were recognized (A, B and C) with antibodies specific for both carrier molecule (AlMV CP) and fused peptide (TVE) in total extracts from infected leaves as well in purified virus samples. Total extracts (C-(total)) from noninoculated plants did not react with either of the antibodies.

a. NF1RSV. Systemically infected leaf tissue was harvested 14-16 days after inoculation for the analysis of NF1RSV accumulation and assembly into particles. Recombinant virus was purified from infected tissue using standard virus, purification procedures (Welter et. al., 1996, Vaccines: New technologies & applications. Cambridge Healthtech Institute's; Yusibov et. al, 1997, Proc. Natl. Acad. Sci. USA 94, 5784-5788). Samples for the analysis (Western and Coomassie) were taken from crude extracts of infected tissue and from purified recombinant virus. Recombinant protein was detected using monoclonal antibodies for AlMV CP (FIG. 5A) and for the G protein of RSV (FIG. 5B). The monoclonal antibody for AlMV CP reacted with control protein (AlMV CP; FIG. 5A) and recombinant protein of expected size (kD) in crude extracts NF1RSV(total); and in purified virus sample NF1RSV(purified); FIG. 5A. The antibodies for G protein recognized only recombinant protein in crude extracts (NF1RSV, (total); FIG. 5B) and in purified virus sample (NF1RSV, purified); FIG. 5B) and did not react with AlMV CP alone. The proteins in crude extracts from non-inoculated plants did not bind either of the 10 antibodies (C, total); FIGS. 5A and B). The FIG. 5C is a Coomassie staining of proteins from crud extracts before virus purification (NF1RSV, total and C, total) from isolated virus sample which shows the effectiveness of purification procedure.

### EXAMPLE 5

### Production of Foreign Proteins Cloned into AlMV RNA3 using Transgenic Plants Expressing the Replicase Proteins of AlMV

Rep plants were inoculated with in vitro transcripts of P3/GFP, P3/gp53, P3/17-1ACH and GFP/CP as described in Example 4. Expression of each recombinant protein in upper systemically infected leaves was assessed by Western and Northern analysis.

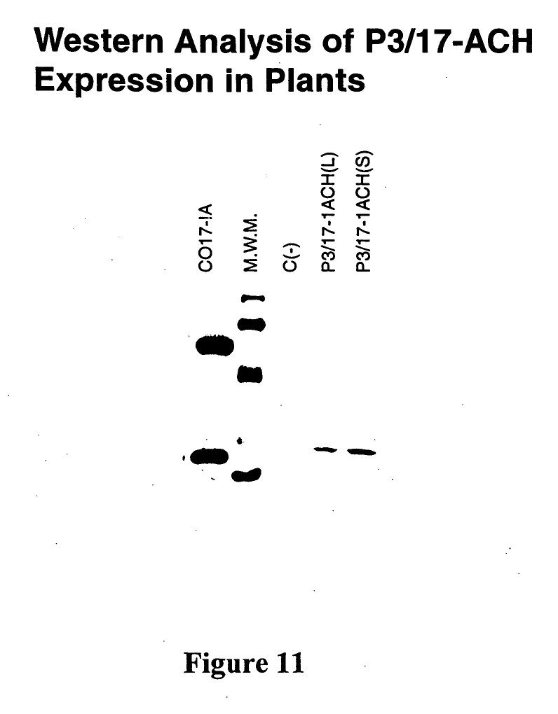

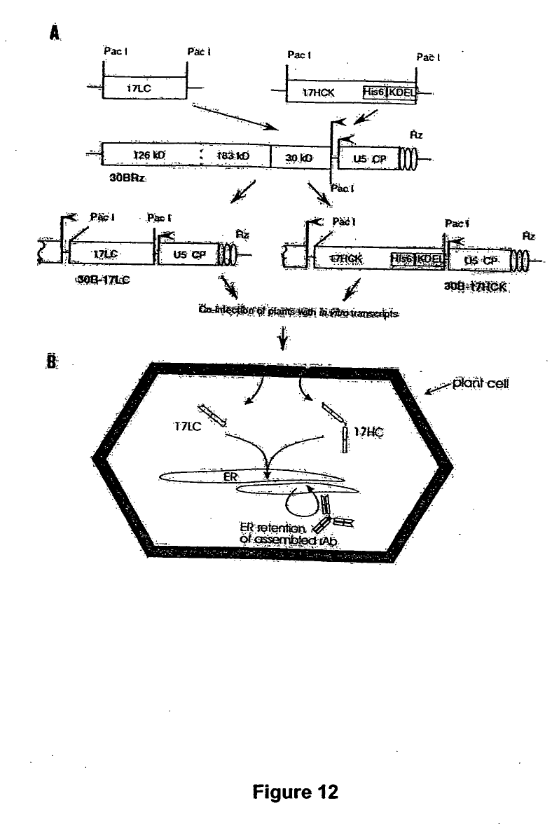

a. P3/17-1 ACH. The plants were inoculated with the 1:1 mixture of in vitro transcripts of P3/17-1ACH and RNA3. Within 14 days after inoculation the systemically infected tissue was analyzed for the accumulation of AlMV CP and 17-1ACH. Western analysis of the extracts from systemically infected tissue contained both AlMV CP and 17-1 ACH (FIG. 11). The proteins were detected using specific antibodies. Monoclonal antibodies for AlMV CP were used to detect the coat protein of virus which is indicative of virus replication and movement. The IgG (peroxidase conjugate) was used to detect the 17-1 ACH. This demonstrates that foreign proteins can be produced using AlMV transcomplementation system.

b. P3/gp53. The plants were inoculated with the 1:1 mixture of in vitro transcripts of P3/gp53 and RNA3. 14 days after inoculation locally and systemically infected leaves were analyzed for the accumulation of AlMV CP and P3/gp53. The antibodies for both AlMV CP and P3/gp53 recognized right size proteins. In addition, we purified the virus from systemically infected leaves and used it for isolation of virus RNA. The isolated virus RNA was used for Northern analysis to test if the recombinant RNA P3/gp53 and its subgenomic RNA consisting of gp53 ORF and RNA3 3′ noncoding region are encapsidated. The minus sense RNA of gp53 was used as a probe.

### EXAMPLE 6

### Construction of TMV Vector for the Production of Foreign Proteins by Transcomplementing the Long Distance Movement Function of Virus

Our hypothesis was to support the systemic movement of defective TMV and produce foreign proteins by transcomplementing this function from another construct.

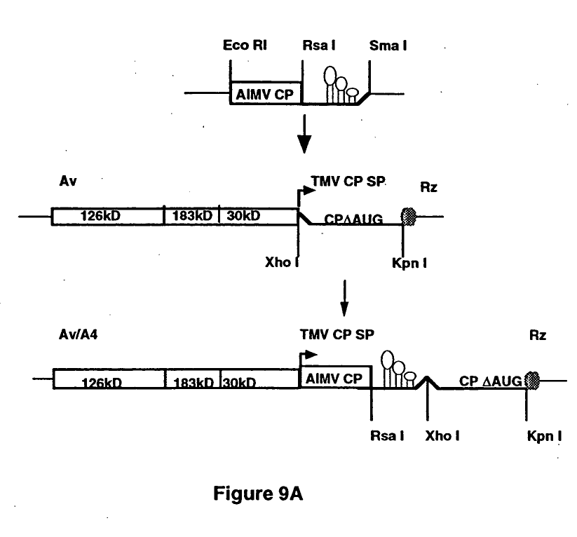

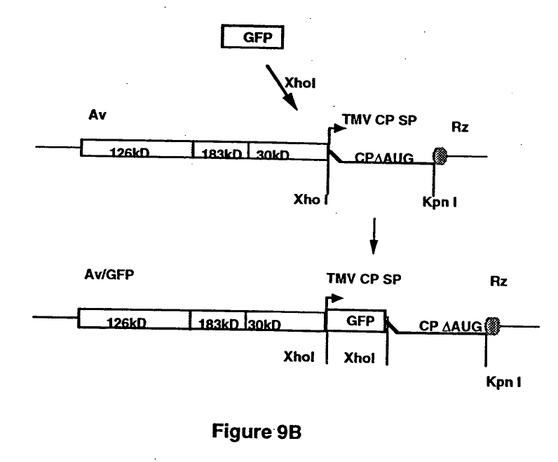

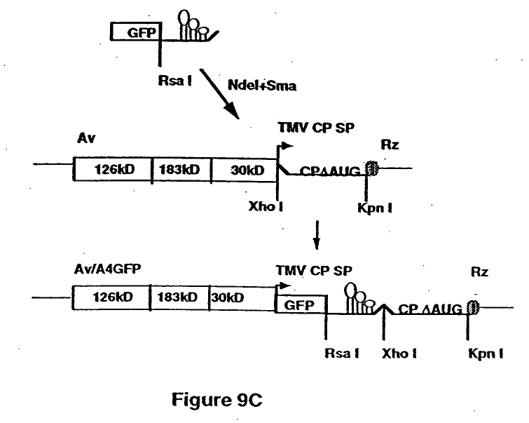

Shown in FIGS. 9A-C are schematic representations of the genome of Av (derivative of TMV) and construction of Av/A4 (A), Av/GFP (B), and Av/A4GFP (C): the 126 kD and 183 kD proteins are required for the TMV replication, 30 kD protein is the viral movement protein, and CP is viral coat protein. Arrow under “TMV CP SP” indicates the subgenomic promoter of TMV CP. ______ is the 3′ noncoding region of AIMV. Rz—indicates ribozyme for self-cleavage.

Av is a construct which is a derivative of TMV. In this construct the translation start codon (ATG) of TMV CP have been replaced with AGA creating a virus defective in production of coat protein. In addition, 42 nucleotides downstream of mutated ATG codon multiple cloning sites Pac I, Pme I, Age I and Xho I were introduced. Av (FIG. 9A) contain full-length TMV defective in coat protein production. To construct the chimeric Av containing AlMV CP we used pSP65A4 (Loesh-Fries et. al, 1985, Virology 146, 177-187) containing full length cDNA of AlMV RNA4. pSP65A4 was digested by EcoR I+Sma I to cleave the DNA fragment containing 5′- and 3′-non coding regions in addition to the open reading frame of A l MV CP. The EcoR I Sma I fragment was blunt ended and cloned into Av linearized by Xho I to create Av/A4 (FIG. 9A). In vitro synthesized transcripts of Av and Av/A4 were used to inoculate the leaves of Nicotiana benthamiana, Nicotiana tabacum MD609 and Spinacia oleracea. Ten days after inoculation samples from locally and systemically infected tissue were analyzed by immunoblot using monoclonal antibodies specific for AlMV coat protein. The coat protein of AlMV was detected both in locally and systemically infected leaves (data not shown). Plants inoculated with in vitro transcripts of Av developed symptoms only on locally inoculated leaves and virus did not move into systemic tissue. Chimeric virus was purified from systemically infected leaves of Nicotiana benthamiana, Nicotiana tabacum MD609 and Spinacia oleracea. Polyethyleneglycol precipitated samples were analyzed for the presence of virus particles and AlMV CP. Electron microscopy revealed the presence of rod shaped particles (300 nm in length) similar to that of TMV. Western analysis and Coomassie staining of SDS-PAGE demonstrated the presence of 24 kD protein recognized with monoclonal antibodies for AlMV CP in purified virus particles. This suggests that the genomic RNA of TMV is encapsidated with AlMV CP. We were not able to detect the TMV CP in these samples neither by Western analysis nor by Coomassie staining of SDS-PAGE (data not shown). These experiments demonstrate that the AlMV CP supports long distance movement of TMV and encapsidates TMV RNA into stable, purifiable rod shaped particles.

Since the AlMV CP supports the systemic movement of TMV genome, we engineered another Av construct where the GFP was cloned under the control of TMV CP subgenomic promoter. We cleaved GFP (see Example 3) by Kpnl+Apal and cloned it into Av linearized by Xhol using blunt end ligation. In a second construct, to improve the movement functions, we cloned GFP with 5′- and 3′-non coding regions of AlMV CP from A3a/GFP (see FIG. 3 in Example 3). We cleaved the ORF of GFP along with 5′- and 3′-non coding regions of AlMV CP from A3a/GFP by NdeI+Smal and cloned into Av linearized by Xho1 using blunt end ligation to create Av-A3a/GFP (FIG. 9B)

We inoculated N. benthamiana plants with the mixture of in vitro synthesized transcripts from Av/A4+Av−A3a/GFP and Av/A4+Av/GFP. Twelve to 15 days after inoculation we analyzed the systemically (upper) infected leaves for the presence of GFP. The control plants inoculated with Av, Av/A4, A4+Av/GFP, and Av−A3a/GFP only had no detectable accumulation of GFP or its messenger RNA in systemically infected leaves. The accumulation of GFP and its messenger RNA in systemically infected leaves was observed using both Western and Northern -analysis. The protein was detected using GFP specific antibodies (Clontech). The Northern analysis was performed using 700 bp DNA fragment as a probe. The results of analysis shows that the foreign proteins can be produced in virus infected plants using functional complementation.

### EXAMPLE 7

### Experimental Immunization of Mice with NF1/RSV Construct Expressing the 25 Amino Acid Antigenic (Protective) Peptide of RSV G Protein and Challenge with RSV

Eight week old female Swiss-Webster, outbred mice were immunized with 50 μg per dose of recombinant NF1/RSV engineered to express the 25 amino acid antigenic (protective) peptide of RSV G protein. Four immunizations of 0.1 ml were administered intraperitoneally at intervals of 2 weeks each with complete Freunds adjuvant (CFA) at 1:1, volume:volume ratio. An equal quantity of a mixture of wild type AMV was used with CFA as a negative control. Identical peptide (VRS-long), expressed in Escherichia coli (E. coli) and assembled into inclusion bodies, has been used as a positive control. E coli expressed peptide VRS-long has been demonstrated to provide complete protection of immunized mice against RSV. Ten to fourteen days after each immunization, serum samples were obtained from individual mice, and RSV-specific antibody titers were assessed. Antigen-specific antibody analysis of serum was performed using an enzyme-linked immunoabsorbant assay (ELISA). ELISA plates (Nunc Polysorp, Denmark) were coated with 100 μl per well of G protein (5 μg/ml in phosphate-buffered saline) overnight at room temperature (RT; about 25 ° C.). Coated plates were washed 3 times with PBS-Tween (0.05%) and then blocked with 5% dried milk in PBS at RT for at least one hour. A series of dilutions of sera were added to the plates (30 μl/well) for 2 to 4 hours at RT. The plates were then washed three times with PBS-Tween and peroxidase conjugated secondary antibodies (goat anti-mouse IgG, either whole molecule or gamma chain specific), were added (100 μl per well) at a final dilution of 1:2000 in PBS, for 1 hour at RT. Plates were the washed 5 times with PBS-Tween and TMB substrate was added (100 μl/well) in phosphate-citrate buffer containing urea, for 30 min at RT in the dark. The reaction was stopped with 2M H₂SO₄ (50 μl per well), and the color change resulting from bound specific antibody measured at 450 nM in an ELISA plate-reader (BioTek, Winooski, Vt.). The titers are shown in Table 1.

After the last immunization the mice were internasally challenged with RSV strain A. Then the mice were sacrificed, and the virus load was monitored. While the mice immunized with backbone vector AlMV had a high load of virus, the mice immunized with NF1/RSV and VRS-long were protected (Table 1).

TABLE 1
ELISA titers of sera from mice immunized i.p. with NF1/RSV
and challenge infection of these mice with RSV.

| | | CHALLENGE |
|---|---|---|
| | | with RSV Proctection |
| Group of mice | ELISA | Test: DICT₅₀log₁₀/g |
| Mice immunized with | 4,800 | \overline{x}: 2.0 |
| NF1/RSV | | |
| Mice immunized with | 300 | \overline{x}: 2.9 |
| A1MV | | |
| VRS-long | 102400 | \overline{x}: 1.7 |

### EXAMPLE 8

### Engineering and Expression of Full-Length Monoclonal Antibody C017-1A Associated with Colorectal Cancer

Light (LC) and Heavy chains (HC) of CO17-1A were cloned into RNA3 of AlMV. The proteins were expressed using the complementation system described above (Examples 3 and 4). The LC was cloned into RNA3 to replace the coat protein gene and to create A3a17-1ALC (FIG. 3B). The HC was cloned into RNA3 to replace the P3 gene and to obtain A3b17-1AHC.

Plant Inoculation and Protein Extraction: Leaves of Rep plants were co-inoculated with in vitro transcription products of recombinant A3a17-1ALC and A3b17-1AHC as described (39) after adding 1 vol (v/v) of FES buffer [sodium-pyrophosphate 1% (w/v), malacoid 1% (w/v), celite 1% (w/v), glycine 0.5 M, K2HPO4 0.3 M, pH 8.5, with phosphoric acid. The mixture of in vitro transcription products and FES buffer was applied to tobacco leaves after abrading the leaf surface with carborundum (320 grit; Fisher, Pittsburgh, Pa.). Inoculation was affected by gentle rubbing to spread the inoculum and further abrade the leaf surface.

Two to three weeks after inoculation, systemically infected leaves were harvested and total soluble protein was extracted by grinding the leaves in 2 vol (w/v) of PE buffer (0.2 M Tris, 5 mM EDT, 0.1% Tween 20). After sedimentation of cell debris in a microtube centrifuge (15 min, 13,000 rpm, 4° C.), the supernatant was used for antibody detection by ELISA.

Purification of rAB CO17-1A: Systemically infected leaves were harvested, homogenized in 1 vol (w/v) of phosphate buffer (0.02 M, pH 7.4) and centrifuged (30 min, 14,000 rpm, 4° C.) to remove debris. The supernatant was additionally purified by filtering through a nitrocellulose membrane (0.45 μm pore size). The final extract was applied at 1-ml/min on a 1-ml Sepharose HiTrap AE protein column (Pharmacia, Piscataway, N.J.) equilibrated with phosphate buffer. The column was washed with 10 vo, (v/v) of phosphate buffer and bound antibody was eluted in 5 vol (v/v) of citrate buffer (0.1 M, pH 4) at 1 ml/min. Fractions of 1, 2, and 3 ml were obtained and analyzed by immunoblot and ELISA.

ELISA: Full-length, assembled rAB was not in the list of references detected by ELISA (Yusibov et. al., 1997, Proc. Natl. Acad. Sci. USA 94, 5784-5788). Buffers were prepared as described in Clark et al., 1977. High binding, 96-well ELISA plates (Nunc, F) were coated with Ag GA 733-2 (Dr. D. Herlyn, Wistar Institute, Philadelphia, Pa.) at a concentration of 1 μg/ml for 1 h at 37 ° C. Plant extract (antibody) was applied in extraction buffer (0.2 M Tris, 1 mM EDTA, 0.1% sodium azide, pH 7.5) and incubated for 2 h at 37 C. Bound rAB CO17-1A was detected using an anti-mouse IgG peroxidase conjugate (whole molecule of Fc specific, Sigma, St. Louis, Mo.).

Expression and Assembly of rAB CO17-1A: Infected leaves of Rep plants were homogenized in extraction buffer, centrifuged to remove cell debris, and directly applied on ELISA plates coated with Ag GA733-2. Plant-produced rAB CO17-1A bound to the Ag was detected using an anti-mouse IgG peroxidase conjugate (whole molecule or Fc). Extracts from plants infected with either A3a17-1ALC or A3b17-1AHC were used as negative controls.

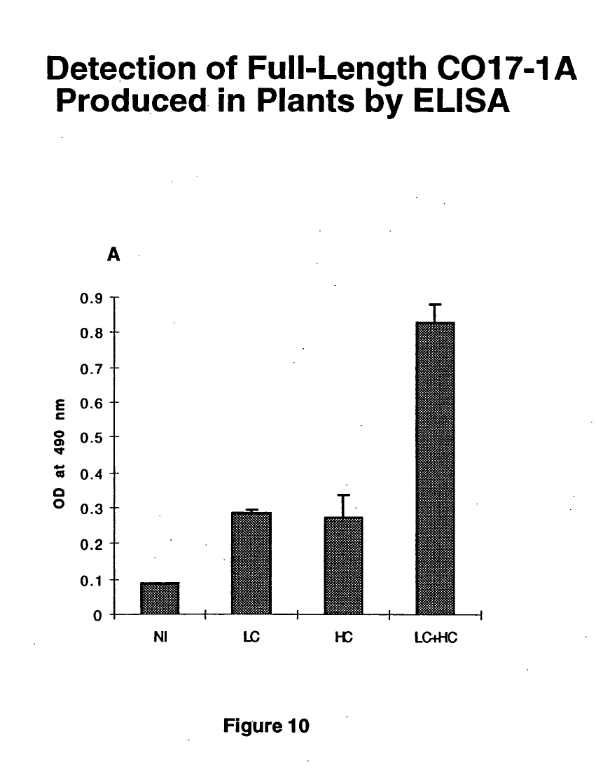

Shown in FIG. 10 is ELISA analysis of CO17-1A self-assembly in virus infected plants. At 19 days post-inoculation, systemically infected plant leaves were homogenized in 2 vol (w/v) of extraction buffer, centrifuged to remove cellular debris, and the supernatant (1:2 dilution) was applied on ELISA plates coated with purified Ag GA733-2. Reactivity of the antibody with GA733-2 was detected with anti-mouse IgG peroxidase conjugate, using the whole molecule (A). NI, extract from non-inoculated control plants; LC and HC, extract from plants expressing only CO17-1A light chain (LC) or heavy chain (HC); LC+HC, extract from plants expressing compete rAb CO17-1A. Data are mean ELISA results from 5 individual plants.

Standard deviation (SD) and significance of differences (p) were calculated. Differences were considered significant at p=0.05.

As shown in FIG. 10, the average OD490 nm reading in samples expressing whole antibody (HC+LC) was 2- to 3-fold higher than that of controls (NI, HC, LC) using the whole molecule.

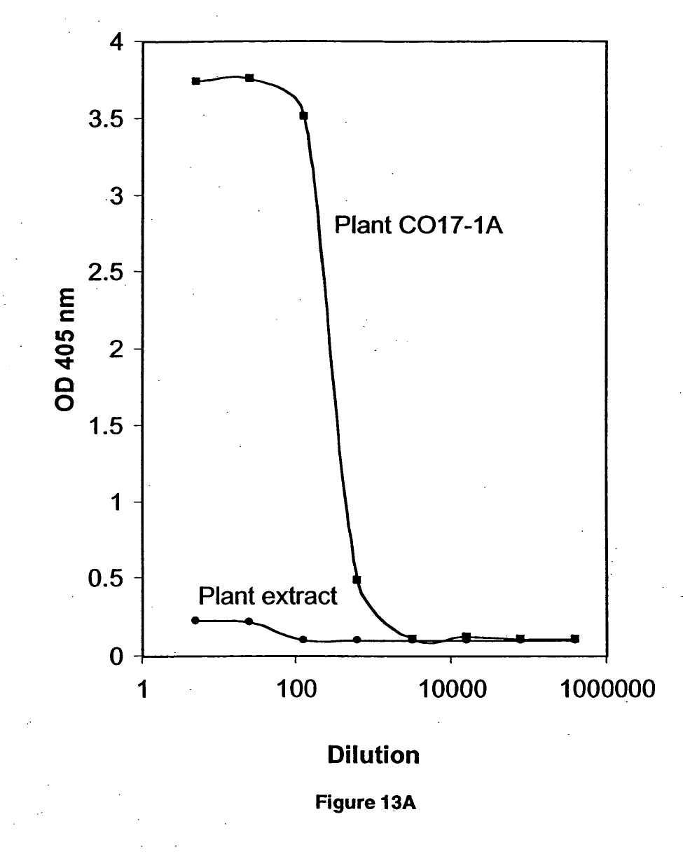

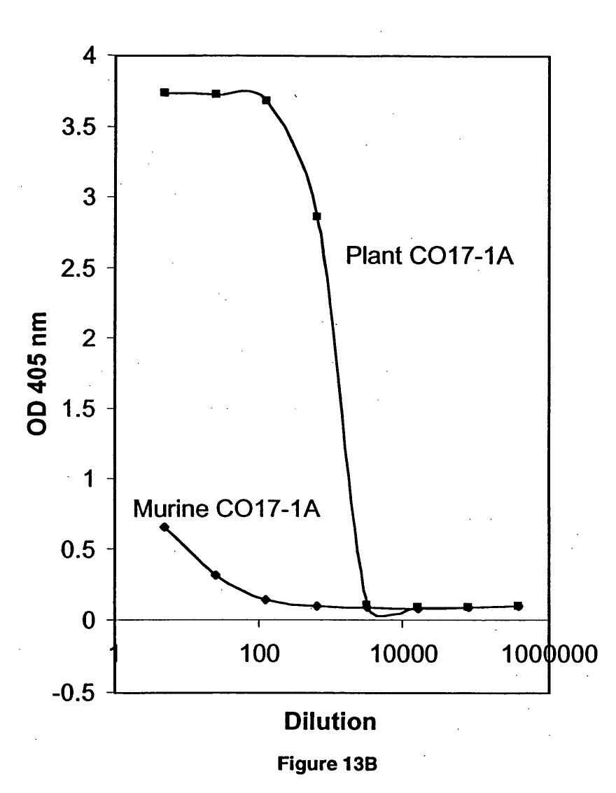

Light (LC) and Heavy chains (HC) of CO17-1A were also cloned into a TMV vector. Shown in FIG. 13 is the schematic representation of cloning (A) of genes encoding HC and LC of rAb CO17-1A and their assembly (B) in infected plant cells. PCR-amplified cDNAs of mAb CO17-1A light chain (17LC) and heavy chain (17HCK) were cloned into TMV vector 30B. The genome of 30B encodes the 126 kDa and 183 kDa proteins required for TMV replication, the 30 kDa protein for virus cell-to-cell movement, and the U5 coat protein (CP) from strain TMV U5. To flags indicate the subgenomic promoter of TMV CP and U5 CP, respectively. Rz indicates ribozyme for self-cleavage of in vitro transcripts. His6 is the protein purification tag. 30B-17LC and 30B17HCK are viruses engineered to express LC and HC of CO17-1A. Upon co-infection of plant cells with in vitro-produced TMV 30B transcripts containing 17 LC and 17HCK, respectively, both chains were expressed, assembled into full-length antibody.

Monoclonal antibody (mAb) CO17-1A (Koprowski et. al., 1979. Somatic Cell Genetics 5: 957-71) is directed against the colorectal cancer-associated antigen (Ag) GA733-2 (Linnenbach et. al., 1989. Proc. Natl. Acad. Sci. USA 86: 27-31), specifically distinguishing between cancer and normal epithelial cells. Genes encoding heavy and light chains (HC and LC) of mAb CO17-1A were expressed from independent viral vector constructs. Upon co-infection of Nicotiana benthamiana plants with in vitro synthesized transcripts of recombinant plant virus cDNA containing genes for HC and LC, full-length rAb CO17-1A was detected in systemically infected leaves 2-3 weeks after inoculation. Recognition of the plant-produced rAb CO17-1A by whole molecule and Fc IgG conjugate indicate that the antibody was correctly assembled. Western blot analysis of the combinant antibody concentrated from plant extracts using a protein A affinity column revealed two bands (25 and 50 kD), similar in size to that of commercially obtained CO17-1A (Centocor, Malvern), indicating glycosylation of the plant-produced antibody. Thus, presence of full-length rAb chains, assembly and binding to the corresponding Ag GA733-2, have been demonstrated in ELISA and by Western immunoblot.

Virus infection of plant tissue has several advantages over the use of transgenic plants for the production of antibody. First the long regeneration time required in plant transformation is not an issue. Second, different host plants can be infected by the same virus vector, allowing time-efficient screening for recombinant gene expression. Third, the time-consuming crossing required for transgenic plants to produce multi-subunit proteins such as secretory antibodies is not necessary. In this study we demonstrate for the first time the use of plant virus vector to produce a full-length antibody in plants.

Plant produced CO17-1A had higher affinity to the corresponding antigen (GA733) then cell culture produced CO17-1A (Centacor) as described in the Example below. Deglycosilation of deglycosilation of cell culture produced CO17-1A (Centacor) increased the binding of this molecule to the antigen. This affinity, however, is still significantly lower than the affinity of plant produced antibody.

All DNA cloning and cell transformations were performed according to Sambrook et. al. 1989, Molecular Cloning, 2nd edn. Cold Spring Harbor Laboratory Press, New York. Esherichia coli DH5α (Life Technologies, Gaithersburg, Md.) and JM109 (Promega, Madison, Wis.) competent cells were used for transformation (cDNA clones of mAb CO17-1A HC and LC were kindly provided by Dr. Peter Curtis, Wistar Institute, Philadelphia, Pa.) The gene for the HC of CO17-1A was PCR-amplified using 5′TTAATTAAGGCCAGATCGGCCATGGAATGGAGCAGAGTCTTT3′ as first-strand primer and 5′TTAATTAAGCGGCCGCTTAGTGATGGTGATGGGTAGGATCGATTTTACCC GGAGTCCGGGAGAA3′ as second-strand primer. Similarly, the DNA encoding the LC of mAb CO17-1A was PCR-amplified using 5′TTAATTAAGGCCAGATCGGCCATGGGCATCAAGATGGGATCA3′ as first-strand primer and 5′TTAATTAAGCGGCCGCCTAACACTCATTCCTGTTGAA3′ as second-strand primer. The PCR products were cloned into the PGEM-T vector (Promega, Madison, Wis.) and the sequences were confirmed (Nucleic Acid Facility, Thomas Jefferson University). The genes for CO17-1A HC and LC were independently cloned into the TMV 30B vector (kindly provided by Dr. William Dawson, University of Florida) using the Pacl-restriction site.

Full-length, assembled rAb was detected by ELISA (Yusibov et. al., 1997). Buffers were prepared as described (Clark and Adams, 1977). High binding, 96-well ELISA plates (Nunc. F) were coated with Ag GA733-2 (kindly provided by Dr. D. Herlyn, Wistar Institute, Philadelphia, Pa. at a concentration of 1 μ.g/ml for 1 h at 37° C. Plant extract (antibody) was applied in extraction buffer (0.2 M Tris, 1 mM EDTA. 0.1% sodium azide, pH 7.5) and incubated for 2 h at 37° C. Bound rAb CO17-1A was detected using an anti-mouse 1 gG peroxidase conjugate (whole molecule or Fc specific, Sigma).

Recombinant proteins expressed in virus-infected plants were analyzed by Western blot (Yusibov et. al., 1997). Proteins from plant extracts were separated electrophoretically on SDS-polyacrylamide gels and electroblotted onto a nylon membrane. After blocking with TBS+0.1% Tween 20. HC and LC were detected using the Vectastain ABC kit (Vector Laboratories, Burlingame, Calif.).

cDNA clones of mAb CO17-1A HC and LC were PCR-amplified introducing restriction sites for Pacl at the 5′- and 3′-ends. Sequences encoding six histidine residues (His6) and a Lys-Asp-Glu-Leu (KDEL) were added in the reading frame of HC at the 3′ end of the gene. His6 is a purification tag and retention of protein in endoplasmic reticulum (ER) by KDEL has been shown to increase yields of recombinant protein. The PCR-amplified DNA was ligated into bacterial vector pGEM-T for subsequent sequence confirmation. The genes encoding CO17-HC and CO17-LC were then cloned into viral vector 30B, under the control of the subgenomic promoter for TMV coat protein mRNA, using the Pacl restriction site to obtain 30B-17HCK and 30-B-17LC.

Infected leaves of N. benthamiana were homogenized in extraction buffer, centrifuged to remove cell debris, and directly applied on ELISA plates coated with Ag GA733-2. Plant-produced rAb CO17-1A bound to the Ag was detected using an anti-Mouse 1 gG peroxidase conjugate (whole molecule or Fc). Extracts from plants infected with either 30B-17LC or 30B-17HCK were used as negative controls. As shown in FIG. 2, the average O.D._{490 am} reading in samples expressing whole antibody (HC+LC) was 2- to 3-fold higher than that of controls (NI, HC, LC) using either the whole molecule for Fc-specific IgG conjugate for detection.

Immunoblot analysis of affinity-purified extracts from plants co-infected with 30B-17LC and 30B-17HCK revealed two bands of approximately 25 and 50 kDa, corresponding in size to LC and HC of tissue-culture produced mAb CO17-1A. Non-infected plant extracts concentrated using protein A affinity column under identical conditions revealed no bands.

By this experiment the expression and assembly of full-length HC and LC to form complete rAb CO17-1A in plants has been demonstrated. The HC and LC were cloned and expressed independently using two viral vector constucts. Recognition of the plant-produced rAb CO17-1A by whole molecule and Fc IgG conjugate indicate that the antibody was correctly assembled. Western blot analysis of the recombinant antibody concentrated from plant extracts using a protein A affinity column revealed two bands (25 and 50 kDa), similar in size to that of commercially obtained CO17-1A (Centocor, Malvern), indicating glycosylation of the plant-produced antibody. The type and extent of glycosylation remains to be determined. Thus, presence of full-length rAb chains, assembly and binding to the corresponding Ag GA733-2, have been demonstrated in ELISA and immunoblot.

Virus infection of plant tissue has several advantages over the use of transgenic plants for the production of antibody. First, the long regeneration time required in plant transformation is not an issue. Second, different host plants can be infected by the same virus vector, allowing time-efficient screening for recombinant gene expression. Third the time-consuming crossing required for transgenic plants (Ma et. al., 1995, Science 268, 716-719) to produce multi-subunit proteins such as secretory antibodies is not necessary. The plant virus vector system has been used for the expression of variety of protein products. In this study we demonstrate for the first time the use of plant virus vector to produce a full-length antibody in plants. Animal trials are currently being carried out to confirm the tumor-suppressive activity of plant-produced rAb CO17-1A.

Monoclonal antibody (mAb) CO17-1A (Koprowski et. al., 1979. Somatic Cell Genetics 5: 957-71) is directed against the colorectal cancer-associated antigen (Ag) GA733-2 (Linnenbach et. al., 1989. Proc. Natl. Acad. Sci. USA 86: 27-31), specifically distinguishing between cancer and normal epithelial cells. Genes encoding heavy and light chains (HC and LC) of mAb CO17-1A were expressed from independent viral vector constructs. Upon co-infection of Nicotiana benthamiana plants with in vitro synthesized transcripts of recombinant plant virus cDNA containing genes for HC and LC, full-length rAb CO17-1A was detected in systemically infected leaves 2-3 weeks after inoculation. Recognition of the plant-produced rAb CO17-1A by whole molecule and Fc IgG conjugate indicate that the antibody was correctly assembled. Western blot analysis of the combinant antibody concentrated from plant extracts using a protein A affinity column revealed-two bands (25 and 50 kD), similar in size to that of commercially obtained CO17-1A (Centocor, Malvern), indicating glycosylation of the plant-produced antibody. Thus, presence of full-length rAb chains, assembly and binding to the corresponding Ag GA733-2, have been demonstrated in ELISA and by Western immunoblot.

Virus infection of plant tissue has several advantages over the use of transgenic plants for the production of antibody. First the long regeneration time required in plant transformation is not an issue. Second, different host plants can be infected by the same virus vector, allowing time-efficient screening for recombinant gene expression. Third, the time-consuming crossing required for transgenic plants to produce multi-subunit proteins such as secretory antibodies is not necessary. In this study we demonstrate for the first time the use of plant virus vector to produce a full-length antibody in plants.

Plant produced CO17-1A had higher affinity to the corresponding antigen (GA733) then cell culture produced CO17-1A (Centacor) as described in the Example below. Deglycosilation of deglycosilation of cell culture produced CO17-1A (Centacor) increased the binding of this molecule to the antigen. This affinity, however, is still significantly lower than the affinity of plant produced antibody.

### EXAMPLE 9

### Comparison of Human Colorectal Cancer Associated Antibody CO17-1A Produced in Plants to that of Cell Culture

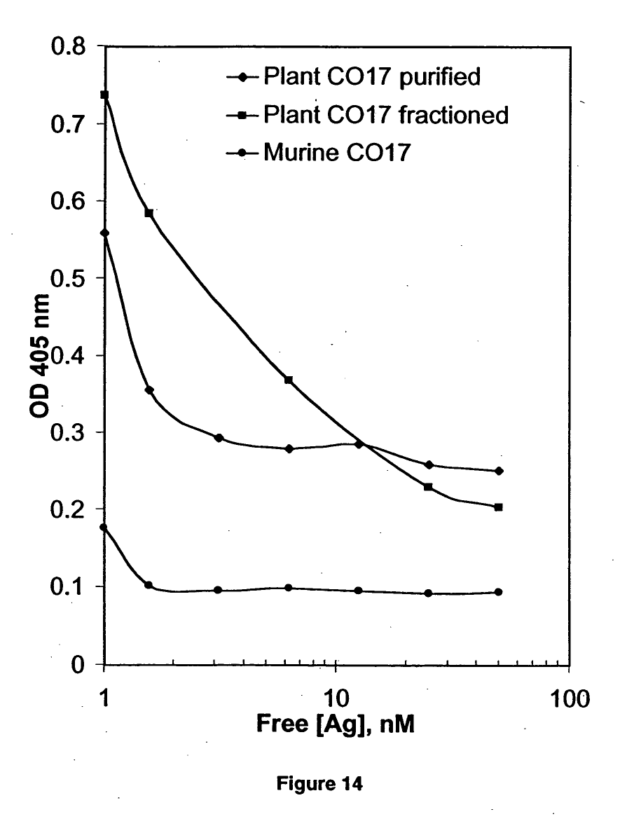

A. Competitive binding. Competition ELISA was performed similar to ELISA experiments described in Example 8. Briefly, 96-well microplates coated with GA733 (2 μg/ml) were incubated with twofold serial dilutions of GA733 (1-100 nM) together with murine or plant antibodies (at constant concentrations indicated in FIGS. 13 and 14). Detection of antibody binding to the solid-phase antigen was performed by incubation with goat anti-mouse IgG-alkaline phosphatase conjugate followed by—nitrophenyl phosphate. The relative avidities of plant and murine antibodies were estimated by calculating the concentration of free antigen required to inhibit+antibody binding by 50% (IC₅₀)—indicated by intercepted lines on the graph (FIG. 14). Approximately 5-fold higher antigen concentration was required to inhibit binding of murine mAb733 to GA733 by 50% (IC₅₀=35 nM) compared with the plant expressed CO17-1A (both purified and fractionated IC₅₀=6.3 nM). IC₅₀ for murine CO17-1A was not estimated due to weak antibody binding. Presented result indicates higher affinity of plant produced antibody to the antigen. Since, the effecacy of interaction between antibody and antigen is important for cancer immunotherapy plant produced antibody may have an advantage over cell culture one due to higher affinity.

B. Antibody affinity measurements by surface plasmon resonance on Biacore. Antibody affinities were measured using the Biacore-X system (Biacore AB, Sweden). Approximately 500 resonance units (RU) of GA733 antigen (100 nM in HBS (10 mM HEPES pH 7.0, 150 mM NaCl)) were immobilized on a HPA chip using hydrophobic interactions and then followed by 400 RU of casein (0.1 mg/ml in HBS) totaling 900 RU on flow cell 2 (FC2). Control surface on flow cell 1 (FC1) was immobilized with 900 RU of casein. Immobilization flow was 10 μl/min. The binding kinetics of murine CO17 and murine mAbGA733 as well as deglycosylated murine Abs were measured at concentration of 100 nM. Purified plant CO17 and deglycosylated plant CO17 were measured at the same dilution 1:10. Binding was measured at a flow rate of 30 μl/min. After each binding measurement surfaces in both flow cells were regenerated with 1 MnaCl pH 3.0. The signal shown in FIG. 14 is a difference between binding to GA733 surface and to a control one (FC2-FC1), thus, representing specific binding. Association phase for each antibody begins at time 0 sec, when sample is injected, dissociation phase starts at 110-140 sec with injection of running buffer.

Experimental data were analyzed using local fitting with BIAevaluation software 3.0. Model curve fitting was done using 1:1 Langmuir interaction.

Antibody deglycosylation. Murine and plant antibodies were enzymatically deglycosylated using PNGase F to release N-linked oligosaccharides followed by NANase II, GALase III, HEXase I and O-Glycosidase DS to release O-linked oligosaacharides in accordance with non-denaturing protocol from Bio-Rad (Deglycosylation Enhancement Kit Instruction Manual Catalog No. 170-6508).

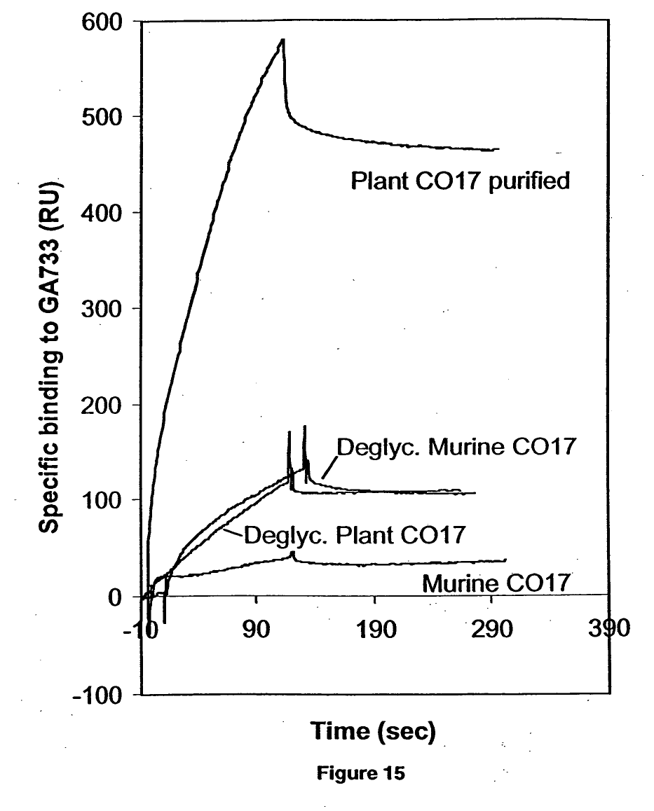

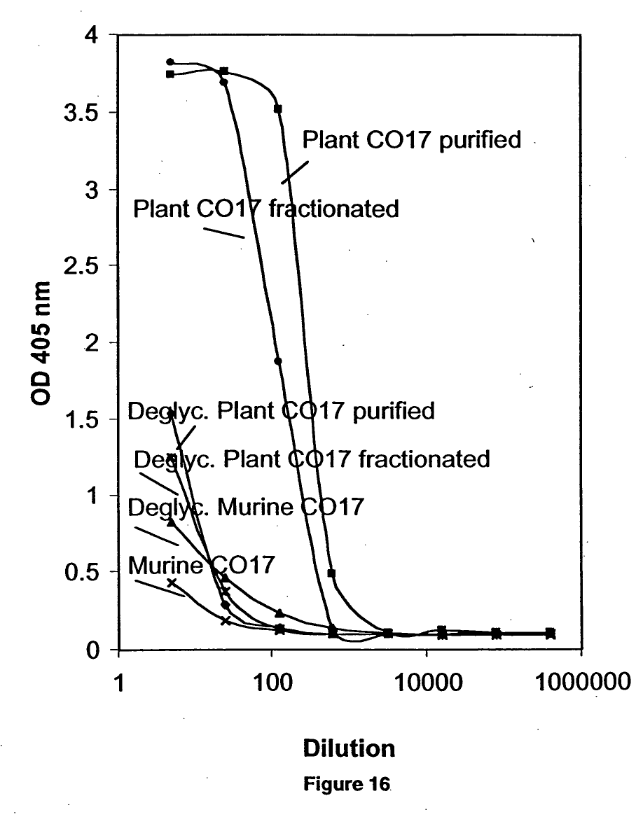

This experiment, once again, demonstrates higher affinity specific binding of plant produced antibody to the corresponding antigen (FIG. 15). In addition, we measured the effect of deglycosilation on antigen binding properties of antibody. As shown on FIG. 15 deglycosilation severely decreased antigen binding properties of plant produced antibody as well as murine GA733 (FIG. 15). However, deglycosilation increased the affinity of cell culture produced CO17-1A. Similar results were obtained by regular ELISA assay too (FIG. 16). These results suggest that plant produced antibody and cell culture produced CO17-1A have different glycosilation profile. These glycons contribute to the overall structure of protein, by that perhaps changing the folding of binding domain of antibody which in its turn effects the affinity of antibody to specific antigen.

Shown in FIG. 13 is ELISA analysis of CO17-1A self-assembly in virus-infected plants. At 19 days post-inoculation, systemically infected plant leaves were homogenized in 2.5 vol (w/v) of extraction buffer, centrifuged to remove cellular debris, and the supernatant (1:2 dilution) was applied on ELISA plates coated with purified Ag GA733. Antibodies bound to GA733 were detected by goat anti-mouse IgG-alkaline phosphatase conjugate in enzymatic reaction with p-nitrophenyl phosphate at 405 nm. Plant produced CO17-1A shows high affinity to the antigen GA733 (A and B) compare to the cell culture produced CO17-1A (B) and control extract from non-inoculated plants (A).

Shown in FIG. 14 is antibodies avidity measurement by competition ELISA. Samples containing different concentrations of GA733 antigen in the fluid phase alongside with constant antibody concentration were applied to the GA733 coated ELISA wells and detected with anti-mouse antibodies conjugated to alkaline phosphatase. ♦—Plant CO17 (1/500 dilution) purified by protein A affinity chromatography; ▪—Plant CO17 (1/500 dilution) partially purified by ammonium sulfate fractionation; ▴—Murine mAbGA733 (0.063 nM); ●—Murine CO17 (0.25 nM). The relative avidities of plant and murine antibodies are estimated by calculating the concentration of free antigen required to inhibit antibody binding by 50% (IC₅₀)—indicated by intercepted lines on the graph. Approximately 5-fold higher antigen concentration was required to inhibit binding of murine mAb733 to GA733 by 50% (IC₅₀=35 nM) compared with the plant expressed CO17 (both purified and fractionated IC₅₀=6.3 nM). IC₅₀ for murine CO17 was not estimated due to weak antibody binding.

Shown in FIG. 15 is the effect of deglycosylation on antibody affinity measured by surface plasmon resonance on Biacore-X. Overlays of sensorgrams showing kinetics of specific binding of indicated antibodies to immobilized GA733 antigen. Approximately 500 resonance units (RU) of GA733 antigen were immobilized on a HPA hydrophobic chip followed by 400 RU of casein (total 900 RU on flow cell 2). Control surface (flow cell 1) was immoblized with 900 RU of casein. Shown signal is a difference between biding to GA733 surface and to a control one, so representing specific binding. Association phase for each antibody begins at time 0 sec, when sample is injected, dissociation phase starts at 110-140 sec with injection of running buffer.

Shown in FIG. 16 is the effect of antibodies deglycosylation measured by ELISA. The wells were coated with 2 mg/ml of GA733 antigen. Samples of indicated antibodies were loaded at the appropriate dilution. Dilutions of murine CO17 (de- and glycosylated) start at 6.25 mM, murine mAbGA733 (de- and glycosylated) at 625 nM, and plant antibodies (de- and glycosylated) were applied undiluted at start. Antibodies bound to GA733 were detected by goat anti-mouse IgG-alkaline phosphatase conjugate in enzymatic reaction with p-nitrophenyl phosphate at 405 nm.

While this invention has been described with a reference to specific embodiments, it will be obvious to those of ordinary skill in the art that variations in these methods and compositions may be used and that it is intended that the invention may be practiced otherwise than as specifically described herein. Accordingly, this invention includes all modifications encompassed within the spirit and scope of the invention as defined by the claims.

1. A method for producing a full-length antibody in a host plant using a virus, comprising:
(a) providing a first recombinant viral vector comprising (1) a recombinant genomic component of the virus having a nucleic acid sequence encoding a movement protein and a nucleic acid sequence encoding a coat protein, and (2) a nucleic acid sequence encoding a heavy chain of the antibody, such that the expression of the recombinant genomic component also results in the expression of the heavy chain of the antibody;
(b) providing a second recombinant viral vector which comprises the same recombinant genomic component as the first recombinant viral vector and a nucleic acid sequence encoding a light chain of the antibody, such that the expression of the recombinant genomic component also results in the expression of the light chain of the antibody; and
(c) infecting the host plant at one or more locations with the first and second recombinant viral vectors, resulting in a systemic infection of the host plant with the first and second recombinant viral vectors,
wherein the heavy and light chains resulting are expressed and assembled into the full-length antibody in the host plant.

2. The method of claim 1, wherein the full-length antibody is a monoclonal antibody.

3. The method of claim 1, wherein the full-length antibody is directed to an antigen selected from the group consisting of hepatitis B surface antigen, enterotoxin, rabies virus glycoprotein, rabies virus nucleoprotein, Norwalk virus capsid protein, gastrointestinal cancer antigen, G protein of Respiratory Syncytial Virus, octreotide, anthrax antigen and colorectal cancer antigen.

4. The method of claim 1, wherein said host plant is a dicotyledon or a monocotyledon.

5. A full-length monoclonal antibody produced in a virus infected plant comprising a heavy chain and a light chain, wherein the heavy chain and the light chain are assembled in planta to form the full-length monoclonal antibody, and wherein the heavy chain results from the expression of a first recombinant genomic component of the virus carrying the heavy chain gene and the light chain results from the expression of a second recombinant genomic component of the virus carrying the light chain gene in said plant.

6. The full-length monoclonal antibody of claim 5, being wherein the full-length antibody is directed to an antigen selected from the group consisting of hepatitis B surface antigen, enterotoxin, rabies virus glycoprotein, rabies virus nucleoprotein, Norwalk virus capsid protein, gastrointestinal cancer antigen, G protein of Respiratory Syncytial Virus, Sandostatin, anthrax antigen or colorectal cancer antigen.

7. The full-length monoclonal antibody produced according to the method of claim 6, wherein the antibody has higher affinity for an antigen than the same antibody produced in a mammalian cell.

8. A method for producing a full-length antibody in a host plant through functional transcomplementation of a virus, the method comprising:
(a) constructing a first recombinant viral vector for infection which comprises a recombinant genomic component of the virus having a movement protein encoding nucleic acid sequence and a coat protein nucleic acid sequence, and a nucleic acid sequence for the heavy chain of the antibody cloned into the recombinant genomic component such that the expression of the recombinant genomic component also results in the expression of the heavy chain of the antibody;
(b) constructing a second recombinant viral vector for infection which comprises the same recombinant genomic component as in step (a) except that a nucleic acid sequence for the light chain of the antibody is cloned into the recombinant genomic component instead of the heavy chain such that the expression of the recombinant genomic component also results in the expression of the light chain of the antibody;
(c) infecting said plant at one or more locations with the first recombinant viral vector and the second recombinant viral vector such that the infection of said plant with the first and second recombinant viral vectors results in systemic infection in said plant,
wherein the first and second recombinant viral vectors are deficient for the virus replicase function, and the host plant is transgenic for expressing replicase genes of a virus to complement the virus replicase function, and the heavy and light chains resulting from the expression of the first and second recombinant genomic components are assembled to a full-length antibody; and
(d) expressing the first and second recombinant genomic components, wherein the heavy and light chains resulting from the expression are assembled into the full-length antibody in the host plant.

9. An isolated full-length antibody comprising a heavy chain and a light chain, wherein the antibody is isolated from a plant tissue containing the full-length antibody produced according to the method of claim 1.

10. A composition comprising the full-length antibody according to claim 9 and a pharmaceutically acceptable carrier.

11. An isolated full-length antibody comprising a heavy chain and a light chain, wherein the antibody is isolated from a plant tissue containing the full-length antibody produced according to the method of claim 8.

12. A recombinant full-length antibody having at least three fold higher binding affinity to the corresponding antigen than the parent antibody.

13. A recombinant full-length antibody having at least six fold higher binding affinity to the corresponding antigen than the parent antibody.

14. A recombinant full-length antibody having at least ten fold higher binding affinity to the corresponding antigen than the parent antibody.

15. A recombinant full-length antibody having at least ten fold higher binding affinity to the corresponding antigen than the parent antibody.

16. A recombinant full-length antibody having a lower dissociation constant than the parent antibody.

17. A method for producing a full-length antibody in a host plant using a virus, comprising:
(a) providing at least a first recombinant viral vector comprising a nucleic acid sequence encoding a viral movement protein, but which lacks a functional nucleic acid sequence for encoding a viral capsid protein, and (2) a heterologous nucleic acid sequence encoding a foreign polypeptide, such that the expression of the viral movement protein also results in the expression of the foreign polypeptide;
(b) providing at least a second recombinant viral vector comprising a nucleic acid sequence encoding a viral capsid protein, but which lacks a functional nucleic acid sequence for encoding a viral movement protein, and (2) a heterologous nucleic acid sequence encoding a foreign polypeptide, such that the expression of the viral capsid protein also results in the expression of the foreign polypeptide;
(c) infecting the host plant at one or more locations with at least the first and second recombinant viral vectors, resulting in a systemic infection of the host plant with the first and second recombinant viral vectors; and
(d) expressing the foreign polypeptides from the first and second viral vectors, wherein the foreign polypeptides expressed by the first and second recombinant viral vectors are either an antibody light chain or an antibody heavy chain, provided that the first and second recombinant viral vectors do not express the same foreign polypeptide,
wherein the expressed antibody heavy and light chains are assembled into the full-length antibody in the host plant.

18. The method of claim 17, wherein the full-length antibody is a monoclonal antibody.

19. The method of claim 17, wherein the full-length antibody is directed to an antigen selected from the group consisting of hepatitis B surface antigen, enterotoxin, rabies virus glycoprotein, rabies virus nucleoprotein, Norwalk virus capsid protein, gastrointestinal cancer antigen, G protein of Respiratory Syncytial Virus, octreotide, anthrax antigen and colorectal cancer antigen.

20. The method of claim 17, wherein the host plant is a dicotyledon or a monocotyledon.

21. The method of claim 17, wherein the viral movement protein expressed by the first recombinant viral vector and the viral capsid protein expressed by the second recombinant viral vector are from different viruses.

22. The method of claim 17, wherein the nucleic acid sequences encoding the viral capsid protein in the first recombinant viral vector are replaced by the nucleic acid sequences encoding the foreign polypeptide.

23. The method of claim 17, wherein the nucleic acid sequences encoding the viral movement protein in the second recombinant viral vector are replaced by the nucleic acid sequences encoding the foreign polypeptide.

24. A method for producing a full-length antibody in a host plant using a virus, comprising:
(a) providing a recombinant viral vector comprising (1) a nucleic acid encoding a viral movement protein and a nucleic acid encoding a viral capsid protein, wherein the viral movement protein and viral capsid protein are from different viruses, and (2) a heterologous nucleic acid sequence encoding at least two foreign polypeptides, such that the expression of the viral movement and viral capsid proteins also results in the expression of the at least two foreign polypeptides;
(b) infecting the host plant at one or more locations with the recombinant viral vector, resulting in a systemic infection of the host plant; and
(c) expressing the at least two foreign polypeptides from the recombinant viral vector, wherein the at least two foreign polypeptides are an antibody light chain and an antibody heavy chain, and wherein the expressed antibody heavy and light chains are assembled into the full-length antibody in the host plant.

25. The method of claim 24, wherein the full-length antibody is a monoclonal antibody.

26. The method of claim 24, wherein the full-length antibody is directed to an antigen selected from the group consisting of hepatitis B surface antigen, enterotoxin, rabies virus glycoprotein, rabies virus nucleoprotein, Norwalk virus capsid protein, gastrointestinal cancer antigen, G protein of Respiratory Syncytial Virus, octreotide, anthrax antigen and colorectal cancer antigen.

27. The method of claim 24, wherein the host plant is a dicotyledon or a monocotyledon.

28. A method for producing a full-length antibody in a host plant using a virus, comprising:
a) providing a first recombinant viral vector comprising a nucleic acid sequence encoding a viral movement protein from a first viral type and a nucleic acid sequence encoding a viral capsid protein from a second viral type;
(b) providing a second recombinant viral vector comprising (1) a nucleic acid sequence encoding the same viral movement protein as encoded by the first recombinant viral vector, but which lacks a functional nucleic acid sequence for encoding a viral capsid protein, and (2) a nucleic acid sequence encoding an antibody heavy chain, such that the expression of the viral movement protein also results in the expression of the antibody heavy chain;
(c) providing a third recombinant viral vector comprising (1) a nucleic acid sequence encoding the same viral movement protein as encoded by the first recombinant viral vector, but which lacks a functional nucleic acid sequence for encoding a viral capsid protein, and (2) a nucleic acid sequence encoding an antibody light chain, such that the expression of the viral movement protein also results in the expression of the antibody light chain;
(d) infecting the host plant at one or more locations with first, second and third recombinant viral vectors, resulting in a systemic infection of the host plant with the first, second and third recombinant viral vectors; and
(e) expressing the antibody heavy and light chains from the second and third viral vectors, wherein the expressed antibody heavy and light chains are assembled into the full-length antibody in the host plant.
